(19) United States
(12) Patent Application Publication
(10) Pub. No.: US 2026/0224502 A1
(43) Pub. Date: Aug. 6, 2026

(54) COMPOSITE PARTICLES

(71) Applicant: UNIVERSITY OF LEICESTER, Leicester, Leicestershire (GB)

(72) Inventors: Sergey PILETSKY, Leicester, Leicestershire (GB); Krzysztof ZALESKI, Leicester, Leicestershire (GB); Alvaro GARCIA CRUZ, Leicester, Leicestershire (GB); Elena PILETSKA, Leicester, Leicestershire (GB); Michael WHITCOMBE, Leicester, Leicestershire (GB)

(73) Assignee: UNIVERSITY OF LEICESTER, Leicester, Leicestershire (GB)

(21) Appl. No.: 19/469,956

(22) PCT Filed: Mar. 27, 2024
(86) PCT No.: PCT/GB2024/050822

§ 371 Date: Sep. 26, 2025

(30) Foreign Application Priority Data
Mar. 29, 2023 (GB) 2304626.1

(51) Int. Cl. A61K 9/50; C08F 20/06; G01N 21/25; G01N 21/31; G01N 21/64; G01N 33/543

(52) U.S. Cl. CPC A61K 9/5089; A61K 9/5021; C08F 20/06; G01N 21/3103; G01N 21/648; G01N 33/54346; G01N 33/54388; G01N 2021/258; G01N 2600/00

(57) ABSTRACT

The disclosure relates to a method of producing a composite particle. The method comprises contacting a catalyst particle, an oxidant and a plurality of monomers. The oxidant is a peroxide, a persulfate or ozone, and the catalyst particle is or comprises a material which possesses Fenton or Fenton-like catalytic activity. The catalyst particle catalyses the disproportionation of the oxidant to create an oxygen-radical species, which then initiates a polymerisation reaction and thereby causes the plurality of monomers to polymerise, and thereby form a polymeric shell around the catalyst particle. The invention extends to composite particles produced by the method, apparatuses comprising the composite particles and uses thereof.

The present invention relates to a method of producing composite particles. The invention extends to the composite particles per se and methods of using them.

Inorganic nanoparticles are technologically important in a number of fields. In many applications, micro and nanomaterials possess properties which are not evident in the materials in the bulk phase, such as tuneable plasmonic resonances, magnetic and optical properties, and others known to those skilled in the art. This leads to their use in diagnostic applications, such as assays (e.g. magnetics bead assays, colorimetric and fluorescent assays, micro array assays, Luminex® Multiplex assays and/or Beckman Coulter assays), surface plasmon resonance (SPR), as well as electrochemical sensors. Nanomaterials and microparticles moreover are able to access otherwise inaccessible sites, such as those within tissues and even cells, due to their small size and mobility.

The applications of unmodified inorganic micro and nanoparticles are, however, limited by a number of factors which have to be overcome in order to exploit the unique properties of these materials.

Micro and nanoparticles maybe prepared by a number of methods. Irrespective of how nanoparticles are prepared, a common feature of unmodified nanomaterials is a strong tendency to aggregate in solution because of their high surface energy and large interfacial surface area. This, together with poor biocompatibility and/or toxicity, linked to their size, shape, and surface chemistry, and poor stability in complex biological environments, limits the potential for the application of nanomaterials in vitro, for example in immunoassays and lateral-flow diagnostics, and in vivo, for example in drug delivery, bioimaging and therapeutic applications.

It is therefore expedient or necessary to coat inorganic micro and nanoparticles with a layer of additional material in order to prevent aggregation, through electrostatic or steric stabilization. Coating the nanoparticle can also allow the surface to be adapted to the specific application for which it is intended to be used. Moreover, the coating may act as a carrier for other components. There are a variety of strategies involving coating micro and nanoparticles, including with silica, lipids, peptides and polymers (Nune S. K., Gunda P., Thallapally P. K., Lin Y.-Y., Forrest M. L., Berkland C. J. Nanoparticles for biomedical imaging. Expert Opin. Drug Deliv., 2009, 6, 1175-1194). The addition of such coatings is a means of providing stabilization against aggregation and for providing substrate functionality suitable for the immobilization of other components, enabling tailored recognition properties or anchoring polymers; in particular organic polymers are preferred in most applications.

There are many ways to coat micro and nanoparticles with robust insoluble polymers for the purpose of changing their physical and chemical properties, and in particular to change their dispersity and prevent aggregation. This may be achieved by treatment of the synthesized nanoparticles with a preformed polymer which partially or completely displaces and replaces the low molar mass stabilizing agents such as citrate or surfactant typically used during the initial nanoparticle synthesis process. In other cases, the polymer forms a secondary shell over the first stabilizing layer, rather than replacing it (Pellegrino T., Manna L., Kudera S., Liedl T., Koktysh D., Rogach A. L., Keller S., Ridler J., Natile G., Parak W. J. Hydrophobic nanocrystals coated with an amphiphilic polymer shell: a general route to water soluble nanocrystals. Nano Lett. 2004, 4, 703-707). Functional core-shell nanoparticles with controlled thickness were prepared by layer-by-layer deposition technique using polymeric polyelectrolytes (Asapu R., Claes N., Bals S., et al. Silver-polymer core-shell nanoparticles for ultrastable plasmon-enhanced photocatalysis. Applied Catalysis B: Environmental. 2017, 200, 31-38). The polymer component maybe strongly adsorbed or chemically bonded to the nanoparticle surface, such that it will not be released into solution, or it maybe cross-linked through physical, photochemical or chemical treatment to form a stable coating. Alternatively, the polymer coating maybe formed in the presence of the nanoparticles by a polymerization process from a monomer, or mixture of monomers, in the presence of the micro and nanoparticles. The latter process however can result in a mixture of products, such as free polymer formed in solution, not associated with nanoparticles, which subsequently requires physical (dialysis), chemical (reduction, precipitation) and/or chromatographic separation techniques to be applied in order to separate the intended product. Moreover, this process precludes the use of some monomers, in particular cross-linkers, as polymer formation in free solution is likely to form an insoluble gel and large composite particles in addition to the intended nanocomposites.

A far better approach is to ensure that the polymer is formed only at, or in close proximity to, the nanoparticle surface. This may be achieved in a number of ways, for example by the use of stabilizing ligands that also function as monomers, capable of polymerization through free radical polymerization, RAFT polymerization, addition polymerization, metathesis polymerization, Diels-Alder reaction, polycondensation or any other process known to those skilled in the art. Alternatively, ligands may be used which combine stabilizing functionality with functional groups capable of initiating polymerization, through thermal, physical, chemical or photochemical activation, such that the locus of polymerization is confined to the nanoparticle surface. However, both of these approaches are limited, as they generally require the synthesis of new reagents that act both as stabilizers and contain the necessary polymerizable or initiating functional groups. This is both expensive and time consuming.

Soluble monomers, or mixtures of monomers, may however be used if the polymerization chemistry is confined to the surface of the micro and nanoparticles through exploiting the physical or chemical properties of the nanomaterial, in some instances with the immobilisation of an otherwise soluble initiator onto the surface of nanoparticles, such that the physical or chemical conditions favouring polymerization are only present at, or in close proximity to, the nanoparticle surface but do extend into the bulk phase. In this case it is possible to use commercially available monomers or monomer mixtures as well as custom synthesized monomers which impart specific properties to the polymer.

Micro and nanoparticles can be physically entrapped into polymers by the “grafting from” process (Pereira S. O., Barros-Timmons A., Trindade T. Polymer@gold nanoparticles prepared via RAFT polymerization for opto-biodetection. Polym., 2018, 10, 189). Grafting of polymers from the surface of nanoparticles can be achieved chemically or photochemically. Activation of the nanomaterials by visible or UV irradiation can trigger polymerization onto their surface (Liu M. S., Peng T. Y., Li H. N., et al. Photoresponsive nanostructure assisted green synthesis of organics and polymers. Appl. Catal. B-Environ. 2019, 249, 172-210). Irradiation with visible light was used to polymerize methyl methacrylate, 2-(tert-butylamino)ethyl methacrylate and ethylene glycol dimethacrylate on the surface of carbon doped TiO₂ nanoparticles (Wang X., Song X., Lin M., et al. Du. Surface initiated graft polymerization from carbon-doped TiO2 nanoparticles under sunlight illumination. Polymer, 2007, 48, 5834-5838; Kong H., Song J., Jang J. Photocatalytic antibacterial capabilities of TiO₂-biocidal polymer nanocomposites synthesized by a surface-initiated photopolymerization. Environ. Sci. Technol., 2010, 44, 5672-5676). The polymers confined at the surface the TiO₂ particles possesses a high thermal stability as shown by TGA analysis. Beyazit et al. reported construction of a cross-linked polymer coating on the surface of up-converting nanoparticles (UCNPs) upon the NIR irradiation (Beyazit S., Ambrosini S., Marchyk N., et al. Versatile synthetic strategy for coating upconverting nanoparticles with polymer shells through localized photopolymerization by using the particles as internal light sources. Angew. Chem. Int. Ed. Engl., 2014; 53, 8919-23). Since the emission from the UCNPs is weak as compared to the direct light, the polymerization proceeds only at the confined, close proximity of the UCNP surface, and thus resulting in the formation of core/shell structure. Ding et al. reported SPR-induced radical polymerization via plasmonic “hot” electrons using Au NPs (Ding T., Mertens J., Lombardi A., et al. Light-directed tuning of plasmon resonances via plasmon-induced polymerization using hot electrons. ACS Photonics, 2017, 4, 1453-1458). It is proposed that upon the absorption of visible photons, due to the strong localized SPR electric fields, hot-electron transfer takes place from Au NP surface to monomers to form initiating free radicals for polymer growth. Photopolymerization is not however feasible for coating nanomaterials which strongly absorb light and prevent irradiation reaching all the particles in the bulk volume.

For these reasons chemical activation of the polymerization reaction at the surface of nanomaterials is preferred to the photochemical option for preparing industrial quantities of polymer-coated nanoparticles. Polymer grafting can benefit from the fact that many known nanomaterials are catalytically active. A variety of nanoscale materials, such as cerium oxide nanoparticles, Au, Pt, Pd nanoparticles, V₂O₅, Fe₃O₄, graphene oxide, and carbon nanotubes, have been discovered to possess catalytic activities (Lin Y., Ren J., Qu X. Catalytically active nanomaterials: a promising candidate for artificial enzymes. Acc. Chem. Res. 2014, 47, 1097-1105). Polymerization can proceed at the surface of catalytic nanoparticles without adding initiators. Thus, Pd nanoparticles were used as active catalysts to polymerize C₂H₂ into trans-polyacetylene, making their surface hydrophobic and preventing aggregation in organic solvents (Dai Y., Liu S., Zheng N. C₂H₂ treatment as a facile method to boost the catalysis of Pd nanoparticulate catalysts. J. Am. Chem. Soc. 2014, 136, 5583-5586).

The formation of nanoparticles with a polymer coating (formed by UV or chemical polymerization) typically requires two separate stages. Thus, all previous examples employ pre-fabricated organic or inorganic nanoparticles. This is not ideal, since it raises question how to prevent aggregation of said nanoparticles prior to their functionalization with a polymer coating. One option to solve this problem is to combine the formation of nanoparticles with polymerization reaction into single-stage process. In one such example noble metals ions were reduced to nanocrystals by the dopamine in redox-oxidation polymerization with HAuCl₄ (Fang Q. L., Zhang J. F., Bai L. F., et al. In situ redox-oxidation polymerization for magnetic core-shell nanostructure with polydopamine-encapsulated-Au hybrid shell. J. Hazard. Mat. 2019, 367, 15-25). This process however is restricted to a very specific class of monomers such as dopamine, aniline, phenol etc. that readily undergo oxidative polymerization.

In accordance with a first aspect of the invention, there is provided a method of producing a composite particle, the method comprising contacting a catalyst particle, an oxidant and a plurality of monomers, wherein the oxidant is a peroxide, a persulfate or ozone, and the catalyst particle is or comprises a material which possesses Fenton or Fenton-like catalytic activity, such that the catalyst particle catalyses the disproportionation of the oxidant to create an oxygen-radical species, which then initiates a polymerisation reaction and thereby cause the plurality of monomers to polymerise, and thereby form a polymeric shell around the catalyst particle.

Advantageously, the method produces a composite particle which maybe used in a number of different applications. The method results in high yields, efficient atom conversion in products, and also a uniform and homogenous population of particles. The method can be adjusted, for instance by selection of the catalyst particle and/or polymer, to produce composite particles with different properties.

Since it is the catalyst particle which catalyses the polymerisation reaction, the polymer will form at, or in close proximity to, the catalyst particle surface. Since the polymer forms around the catalyst particle, it is more uniform than a catalyst particle produced using preformed polymer. Additionally, each catalyst particle will comprise a polymer shell, avoiding the production of clusters of catalyst particle encased in a polymeric coating. Furthermore, the production of free polymer, which is not attached to a catalyst particle, is also avoided.

Additionally, the reaction can be conducted under mild conditions (e.g. at or close to a neutral pH) and can be used to produce a large range of different polymeric shells. The thickness and density of the polymeric shell may be controlled by controlling the time of polymerization and/or controlling the concentration of monomers and/or concentration of the oxidant.

The composite particles of the invention may be known as “nanozymes”, i.e. artificial enzymes that have catalytic activity and are made up of nanomaterials. The composite particle maybe capable of mimicking the functions of natural enzymes and can be designed to be more stable, cost-effective, and efficient than their biological counterparts.

The present composite particles, or nanozymes, have several potential applications in diagnostic assays. For instance, they can be used as labels in diagnostic tests to amplify the signal of target molecules, making it easier to detect them. They can also be used to catalyse reactions in diagnostic assays, such as the conversion of a non-fluorescent molecule into a fluorescent molecule, which can be detected using a fluorescence reader.

The present composite particles, or nanozymes, may be used to catalyst the production of a redox active species. For example, they may be used to catalyse the decomposition of hydrogen peroxide (H₂O₂) into hydroxyl radicals (HO·).

In addition, nanozymes can be used in biosensors for the detection of various biomolecules, such as glucose, cholesterol, and proteins. They can also be used in imaging applications, such as photoacoustic imaging, where they can generate contrast by converting light energy into acoustic waves.

Overall, nanozymes offer a promising platform for the development of new and improved diagnostic tools, with the potential to provide faster, more sensitive, and more accurate diagnosis of various diseases.

While the first aspect defines a method of producing a composite particle, it will be appreciated that it can be used to produce a plurality of composite particles. It will be appreciated that when the method produces a plurality of composite particles, the method will comprise contacting a plurality of catalyst particles, the oxidant and the plurality of monomers. The method may cause a polymeric shell to separately form around each of the plurality of catalyst particles.

The method may comprise contacting the catalyst particle, the oxidant (e,g, peroxide or persulfate) and the plurality of monomers in a solution. The solution may comprise a solvent, which may be water, an organic solvent or a combination thereof. The organic solvent may be an alcohol. The alcohol may be a C₁₋₅ alcohol or a C₁₋₃ alcohol. In some embodiments, the preferred solution is an aqueous solution.

The solution may have a pH between 3 and 12 at 20° C., between 4 and 11 at 20° C., between 5 and 10 at 20° C., between 6 and 9 at 20° C., between 6.5 and 8.5 at 20° C., between 7 and 8 at 20° C. or between 7.4 and 7.6 at 20° C. Advantageously, the method maybe conducted under mild reaction conductions and at a pH which is substantially neutral. It may be appreciated that the temperature of 20° C. has been provided to enable the pH of the solution to be accurately defined. However, the method may be conducted at a range of temperatures, as defined herein. It should be understood that the recitation of temperature here in relation to the pH is not intended to limit the temperature at which the reaction is conducted.

The solution may comprise a buffer. The buffer may be configured to maintain the solution at a desired pH. The desired pH may be as defined above. Suitable buffers are known in the art and any suitable buffer may be used. The buffer may be or comprise a phosphate buffer, an acetate buffer or a borate buffer. The buffer may be provided at a concentration of between 0.05 and 1,000 mM, between 0.1 and 500 mM, between 0.5 and 100 mM, between 1 and 50 mM, between 2.5 and 20 mM, between 5 and 15 mM, between 7.5 and 12.5 mM or between 9 and 11 mM.

The catalyst particle may be understood to possesses Fenton or Fenton-like catalytic activity if it is capable of catalysing the disproportionation of the oxidant to create an oxygen-radical species. The catalyst particle maybe able of catalysing the disproportionation of the oxidant to create two different radical species.

If the peroxide or persulfate was understood to have general formula R—O—O—R, where both R groups are the same or different and are any suitable group to provide a peroxide or persulfate, then the oxygen-radical species may have formula R—O· and R—O—O·. If the peroxide is hydrogen peroxide then the oxygen-radical species may be H—O· and H—O—O·.

The catalyst particle may be understood to form the core of the composite particle.

The composite particle maybe a composite nanoparticle or a microparticle. The composite particle maybe understood to be a composite nanoparticle if it has a dimension or a diameter of less than 1 μm. The composite nanoparticle maybe understood to be a composite microparticle it is has a dimension or a diameter of between 1 μm and 1000 μm. Preferably, the composite nanoparticle is a composite nanoparticle.

The composite particle may have a dimension or a diameter of less than 5,000 nm, less than 2,000 nm, less than 1,500 nm, less than 1,250 nm, less than 1,000 nm, less than 900 nm, less than 800 nm, less than 700 nm, less than 600 nm, less than 500 nm, less than 400 nm, less than 300 nm, less than 200 nm, less than 150 nm or less than 120 nm. The composite particle may have a dimension or a diameter of at least 1 nm, at least 50 nm, at least 100 nm, at least 200 nm, at least 300 nm or at least 400 nm. The composite particle may have a dimension or a diameter of between 1 and 5,000 nm, between 50 and 2,000 nm, between 100 and 1,500 nm, between 150 and 1,250 nm, between 200 and 1,000 nm, between 300 and 900 nm, between 400 and 800 nm. The composite particle may have a dimension or a diameter of between 10 and 2,000 nm, between 30 and 1,000 nm, between 50 and 900 m, between 60 and 800 nm, between 70 and 700 nm, between 80 and 650 nm, between 90 and 640 nm, or between 95 and 630 nm. In some embodiments, the composite particle has a dimension or a diameter of between 10 and 600 nm, between 30 and 500 nm, between 50 and 400 m, between 60 and 300 nm, between 70 and 200 nm, between 80 and 175 nm, between 90 and 150 nm, or between 95 and 130 nm. The dimension or diameter may be measured using dynamic light scattering (DLS) of transmission electron microscopy (TEM).

In embodiments where the method comprises producing a plurality of composite particles, the plurality of composite particles may have an average diameter which is as defined above.

The catalyst particle may be a nanoparticle. For instance, the catalyst particle may have a dimension or a diameter of less than 1,000 nm, less than 900 nm, less than 800 nm, less than 700 nm, less than 600 nm, less than 500 nm, less than 400 nm, less than 300 nm, less than 200 nm, less than 150 nm or less than 120 nm. The catalyst particle may have a dimension or a diameter of at least 1 nm, at least 10 nm, at least 20 nm, at least 30 nm, at least 40 nm, at least 50 nm, at least 60 nm, at least 70 nm, at least 80 nm, at least 90 nm or at least 100 nm. The catalyst particle may have a dimension or a diameter of between 1 and 1,000 nm, between 10 and 900 nm, between 20 and 800 nm, between 25 and 700 nm, between 25 and 600 nm, between 30 and 500 nm, between 30 and 400 nm, between 35 and 300 nm, between 35 and 200 nm, between 40 and 150 nm, between 40 and 120 nm. The catalyst particle may have a diameter of between 1 and 100 nm, between 2 and 80 nm, between 3 and 60 nm, between 4 and 40 nm or between 5 and 20 nm. The dimension or diameter may be measured using dynamic light scattering (DLS) of transmission electron microscopy (TEM).

In embodiments where the method uses a plurality of catalyst particles, the plurality of catalyst particles may have an average diameter, which is as defined above.

In some embodiments, the catalyst particle is unbound. Accordingly, in embodiments where the catalyst particle, the oxidant (e.g. peroxide or persulfate) and the plurality of monomers are contacted in a solution, the catalyst particle maybe provided as a suspension in the solution.

In some embodiments, the catalyst particle is bound to a substrate. The substrate may be a solid substrate. The substrate may comprise or be a metal or metalloid, glass and/or a polymer. The metal or metalloid maybe a pure metal, a pure metalloid, an impure metal, an impure metalloid, an alloy, a metal containing compound or a metalloid containing compound. The substrate may comprise a coating. The catalyst may be bound to the coating.

In one embodiment, the catalyst is bound to a substrate, and the substrate is an optical fibre comprising a polymer coating.

The catalyst particle may comprise or consist of a metal, a metal alloy, a metal oxide, a metal salt, a semiconductor, a two-dimensional material, a fullerene, a carbon nanotube, a quantum dot, a carbon nanodot, a carbide and/or a nitride. The carbon nanotube may be a single walled carbon nanotube or a multi walled carbon nanotube.

The metal maybe gold, silver, platinum, palladium, iron, nickel, zinc, chromium, titanium, zirconium, hafnium, vanadium, niobium, tantalum, molybdenum or tungsten. The metal alloy may comprise iron, copper, chromium, nickel, titanium, zirconium, hafnium, vanadium, niobium, tantalum, chromium, molybdenum and/or tungsten. The metal oxide maybe iron oxide, aluminium oxide, titanium oxide, cerium oxide, zirconium oxide, hafnium oxide, vanadium oxide, niobium oxide, tantalum oxide, chromium oxide, molybdenum oxide or tungsten oxide. The metal salt may be an organic or inorganic salt. The metal salt may be an iron salt, a copper salt, a titanium salt, a zirconium salt, a hafnium salt, a vanadium salt, a niobium salt, a tantalum salt, a chromium salt, a molybdenum salt or a tungsten salt. The semiconductor may be or comprise silicon, gallium or germanium. The two-dimensional material may be graphene, or a graphene derivative, such as graphene oxide. The fullerene may encapsulate an entrapped ion. The entrapped ion may be a metal ion. The quantum dot maybe a carbon quantum dot or a semiconductor quantum dot. The semiconductor quantum dot may comprise or consist of a semiconductor as previously defined. The carbide may be titanium carbide, zirconium carbide, hafnium carbide, vanadium carbide, niobium carbide, tantalum carbide, chromium carbide, molybdenum carbide or tungsten carbide. The nitride may be titanium nitride, zirconium nitride, hafnium nitride, vanadium nitride, niobium nitride, tantalum nitride, chromium nitride, molybdenum nitride or tungsten nitride.

In some embodiments, the catalyst particle comprises or consists of gold. In alternative embodiments, the catalyst particle comprises or consists of iron oxide.

The method may comprise producing the catalyst particle.

The method may comprise a first step of producing the catalyst particle, and a second, separate step of contacting the catalyst particle, the oxidant and the plurality of monomers, as defined above. Accordingly, in this embodiment, the method may be viewed as a two-step or two-pot synthesis. The catalyst particle may be produced using standard methods, which will be known in the art.

The method may comprise stabilising the catalyst particle to prevent aggregation. Accordingly, the method may comprise producing the catalyst particle in the presence of a stabilizing ligand. The stabilising ligand may be a citrate, a phosphate, a sulphate, a borate, an ascorbate, a ketone or a surfactant. The method may comprise displacing the stabilizing ligand prior to or consecutively to contacting the catalyst particle, the oxidant and the plurality of monomers. Alternatively, the plurality of monomers may polymerise, and thereby form a polymeric shell around the catalyst particle and the stabilizing ligand.

In a preferred embodiment, the method comprises producing the catalyst particle and contacting the catalyst particle, the oxidant and the plurality of monomers in a single step. In this embodiment, the method may be viewed as a one-step or one-pot synthesis.

Accordingly, the method may comprise contacting a catalyst particle precursor, the oxidant and the plurality of monomers, wherein the catalyst particle precursor is configured to produce the catalyst particle. The catalyst particle, once it has formed, will contact the oxidant and the plurality of monomers, and will catalyse the disproportionation of the oxidant to create an oxygen-radical species, which then initiates a polymerisation reaction and thereby cause the plurality of monomers to polymerise, and thereby form a polymeric shell around the catalyst particle. Accordingly, the polymerisation reaction may take place spontaneously once the catalyst particle has formed.

It maybe appreciated that the method may comprise contacting the catalyst particle precursor, the oxidant and the plurality of monomers in a solution.

The catalyst particle precursor may comprise a reagent which can be oxidised, reduced or hydrolysed to produce the catalyst particle; and an oxidising agent, a reducing agent or a hydrolysing agent. The reagent which can be oxidised, reduced or hydrolysed to produce the catalyst particle may be an inorganic salt, an acid or a hydroxide. The inorganic salt, the acid or the hydroxide may comprise a metal cation. The metal cation maybe a gold cation, an iron cation, a silver cation, a platinum cation, a palladium cation, a titanium cation, a zirconium cation, a hafnium cation, a vanadium cation, a niobium cation, a tantalum cation, a chromium cation, a molybdenum cation or a tungsten cation. In some embodiments, the cation is an Au³⁺ cation. Accordingly, the acid may be hydrogen tetrachloroaurate (III), or a solvate thereof. The solvate may be hydrogen tetrachloroaurate (III) trihydrate or hydrogen tetrachloroaurate (III) tetrahydrate. The reducing agent maybe an organic compound containing a carboxylic group, a ketone group and/or a hydroxyl group. In some embodiments, the reducing agent is a ketone. The ketone maybe cyclohexanone or acetone.

The metal cation maybe present in the solution at a concentration between 0.0001 and 5 mM, between 0.0005 and 1 mM, between 0.001 and 0.5 mM, between 0.005 and 0.25 mM, between 0.01 and 0.10 mM, between 0.02 and 0.08 mM or between 0.04 and 0.05 mM.

The reducing agent maybe present in the solution at a concentration between 0.005 and 10 M, between 0.01 and 5 M, between 0.05 and 2.5 M, between 0.1 and 1 M, between 0.2 and 0.8 M, between 0.4 and 0.5 M.

The molar ratio of the metal cation to the reducing agent maybe between 1:1 and 1:1,000,000, between 1:100 and 1:100,000, between 1:500 and 1:50,000, between 1:1,000 and 1:25,000, between 1:5,000 and 1:20,000, between 1:7,500 and 1:15,000, between 1:9,000 and 1:12,500 or between 1:9,500 and 1:10,000.

The peroxide may be hydrogen peroxide, a peroxy acid, a main group peroxide or an organic peroxide. The persulfate may be ammonium persulfate.

In some preferred embodiments, the oxidant is hydrogen peroxide.

The oxidant maybe present in the solution at a concentration between 0.005 and 10 M, between 0.01 and 5 M, between 0.05 and 2.5 M, between 0.1 and 1 M, between 0.2 and 0.8 M or between 0.4 and 0.5 M.

The plurality of monomers are preferably a plurality of monomers capable of undergoing radical addition polymerisation. Accordingly, the plurality of monomers may be understood to comprise a plurality of molecules, wherein each molecule comprises one or more unsaturated carbon-carbon bonds. It will be appreciated that the phrase “a plurality of monomers” may be used to refer to a plurality of monomer molecules.

The plurality of monomers may consist of a single chemical species. Alternatively, the plurality of monomers may comprise two or more different chemical species.

The plurality of monomers may comprise acrylic acid, an ester and/or an amide of acrylic acid, methacrylic acid, an ester and/or an amide of methacrylic acid, a vinyl ether, a vinyl ester, a vinyl aromatic compound or a combination thereof.

In one embodiment, the plurality of monomers comprise one or more chemical species which comprise a functional group configured to impart a desired property to the polymer.

The functional group maybe configured to enable the attachment of a further species. For instance, the functional group may comprises a carboxylic group, an amine group or a glycidyl group. The functional group may enable the attachment of the further species through chemical reaction or bioconjugation methods, known to those skilled in the art.

The further species may be an inorganic ion, a nucleic acid, a cell, a spore, a virus, a microorganism, a tissue sample, a carbohydrate, a peptide, a protein, a drug, a drug intermediate, a drug precursor, a hormone, a vitamin, a biomarker, a toxin, a pesticide, a herbicide, an explosive, a nerve agent, a pollutant, an endocrine disrupting compound, a nucleotide, a nucleoside, a metabolite, an epitope, an antigen, a receptor, a receptor fragment, an antibody, an antibody fragment, a dye, an indicator, a radionuclide, or any other component known to those skilled in the art. The carbohydrate may be an oligosaccharide or a polysaccharide. The polysaccharide may be a glycosaminoglycan. The protein maybe a nucleoprotein, a mucoprotein, a lipoprotein, a synthetic protein or a glycoprotein. The drug maybe a steroid, an immunosuppressant, a heparin or an antibiotic. The biomarker may be a biomarker of a pathological state or a disease state. The nucleic acid maybe an oligonucleotide, DNA or RNA. The metabolite maybe a drug metabolite and/or a secondary metabolite.

Alternatively, or additionally, the functional may be configured to cause the polymer to be fluorescent, for instance the functional group may comprise Fluorescein, Rhodamine, Dansyl, Texas Red, Alexa Fluor, Cy5 or green fluorescent protein (GFP).

Alternatively, or additionally, the functional may be configured to cause the polymer to be electroactive. For instance, the functional group may comprise an iron derivatives, such as ferricyanide, ferrocyanide or ferrocene, a ruthenium derivative, such as [Ru(NH₃)₆^{]3+/2+}, methylene blue, a porphyrin or a metallocene.

Alternatively, or additionally, the plurality of monomers may comprise one or more chemical species with additional functionality. For instance, the one or more chemical species with additionally functionality may comprise a derivative of amino acids, a nucleoside, a nucleotide, a carbohydrate, styrene or a derivative thereof, acrylamide or a derivative thereof, butadiene, acrylonitrile, vinyl acetate, a vinyl monomer, an allyl monomer, an acetylene, an acrylate, a methacrylate, an acrylamide, a methacrylamide, a chloroacrylate, an itaconate or a trifluoromethylacrylate. A derivative of styrene may be divinyl benzene.

Alternatively, or additionally, the plurality of monomers may comprise a polymerizable dye, or a polymerizable reactive species. Advantageously, the polymeric shell will enable detection, grafting, therapeutic action, diagnostics and additional chemical modification.

Accordingly, the plurality of monomers may comprise one or more chemical species of formula (I):

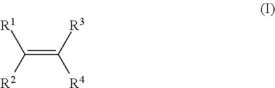

wherein, R¹ to R⁴ are independently H, an optionally substituted alkyl, optionally substituted alkenyl, optionally substituted alkynyl, optionally substituted cycloalkyl, optionally substituted aryl, optionally substituted heterocycyl, optionally substituted heteroaryl, COOR⁵ or CONR⁵R⁶; and
R⁵ and R⁶ are independently H, an optionally substituted alkyl, optionally substituted alkenyl or optionally substituted alkynyl.

The or each alkyl, alkenyl or alkynyl may be an optionally substituted C₁₋₁₂ alkyl, an optionally substituted C₂₋₁₂ alkenyl or an optionally substituted C₂₋₁₂ alkynyl. More preferably, the or each alkyl, alkenyl or alkynyl is an optionally substituted C₁₋₆ alkyl, an optionally substituted C₂₋₆ alkenyl or an optionally substituted C₂₋₆ alkynyl.

The or each alkyl, alkenyl or alkynyl may be unsubstituted or substituted with one or more of oxo, OR⁷, NR⁷R⁸, COOR⁷ OCOR⁷, CONR⁷R⁸ or NR⁷COR⁸, wherein R⁷ and R⁸ are independently H, C₁₋₁₂ alkyl, C₂₋₁₂ alkenyl or C₂₋₁₂ alkynyl.

The optionally substituted cycloalkyl may be optionally substituted C₃₋₁₂ cycloalkyl. The optionally substituted aryl may be optionally substituted C₆₋₁₀ aryl. The optionally substituted heterocycle may be optionally substituted 3 to 12 membered heterocycle. The optionally substituted heteroaryl may be optionally substituted 5 to 10 membered heteroaryl. In multi-ring structures if one ring is aromatic then the entire ring structure may be referred to aryl or, if one of the ring atoms is a heteroatom, heteroaryl. In multi-ring structures if one ring atom is a heteroatom then the entire ring structure maybe referred to as heterocyclic, if none of the rings are aromatic, or heteroaryl, if one or more of the rings are aromatic.

The cycloalkyl, aryl, heterocycle or heteroaryl may be unsubstituted or substituted with one or more of oxo, OR⁷, NR⁷R⁸, COOR⁷ OCOR⁷, CONR⁷R⁸ or NR⁷COR⁸, wherein R⁷ and R⁸ are independently H, C₁₋₁₂ alkyl, C₂₋₁₂ alkenyl or C₂₋₁₂ alkynyl.

Preferably, R¹ is H.

Preferably, R² is H.

Preferably, R³ is H, optionally substituted C₁₋₆ alkyl, optionally substituted C₂₋₆ alkenyl or optionally substituted C₂₋₆ alkynyl. More preferably, R³ is H or C₁₋₃ alkyl, C₂₋₃ alkenyl or C₂₋₃ alkynyl. Most preferably, R³ is H or methyl.

Preferably, R⁴ is COOR⁵ or CONR⁵R⁶.

Preferably, R⁵ and R⁶ are independently H, optionally substituted C₁₋₁₂ alkyl, optionally substituted C₂₋₁₂ alkenyl or optionally substituted C₂₋₁₂ alkynyl. More preferably, R⁵ and R⁶ are independently H, optionally substituted C₁₋₆ alkyl, optionally substituted C₂₋₆ alkenyl or optionally substituted C₂₋₆ alkynyl. Most preferably, R⁵ and R⁶ are independently H, methyl, ethyl, i-propyl, t-butyl, CH₂CH₂OH or CH₂CH₂CH₂NH₂.

Alternatively, R⁴ may be optionally substituted C₃₋₁₂ cycloalkyl, optionally substituted C₆₋₁₀ aryl, optionally substituted 3 to 12 membered heterocycyl or optionally substituted 5 to 10 membered heteroaryl. R⁴ maybe optionally substituted phenyl or optionally substituted 5 or 6 membered heteroaryl. R⁴ maybe pyridine.

Alternatively, R⁴ maybe optionally substituted C₁₋₁₂ alkyl, optionally substituted C₂₋₁₂ alkenyl or optionally substituted C₂₋₁₂ alkynyl. More preferably, R⁴ is optionally substituted C₁₋₆ alkyl, optionally substituted C₂₋₆ alkenyl or optionally substituted C₂₋₆ alkynyl. Most preferably, R⁴ is optionally substituted C₁₋₃ alkyl, optionally substituted C₂₋₃ alkenyl or optionally substituted C₂₋₃ alkynyl. R⁴ maybe CH₂NH₂.

Accordingly, the plurality of monomers may comprise one or more chemical species selected from the group consisting of acrylic acid, acrylamide, methacrylamide, N-isopropylacrylamide, N-tert-butylacrylamide, hydroxyethylmethacrylate, methacrylic acid, N-3-aminopropyl methacrylamide, N,N′-diethyl acrylamide, vinylpyridine and allylamine.

The plurality of monomers may comprise one or more crosslinking agents. The or each crosslinking agent maybe a molecule which comprises two or more unsaturated carbon-carbon bonds. The or each crosslinking agent may comprise a chemical species of formula (II):

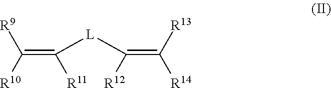

wherein, R⁹ to R¹⁴ are independently H, an optionally substituted alkyl, optionally substituted alkenyl, optionally substituted alkynyl, COOR⁵ or CONR⁵R⁶; R⁵ and R⁶ are independently H, an optionally substituted alkyl, optionally substituted alkenyl or optionally substituted alkynyl; and L is a linker.

The alkyl, alkenyl or alkynyl maybe as defined above.

R⁹ to R¹⁴ are preferably each H, optionally substituted C₁₋₆ alkyl, optionally substituted C₂₋₆ alkenyl or optionally substituted C₂₋₆ alkynyl. More preferably, R⁹ to R¹⁴ are H or C₁₋₃ alkyl, C₂₋₃ alkenyl or C₂₋₃ alkynyl. Most preferably, R⁹ to R¹⁴ are H or methyl.

The linker may be one or more heteroatoms, an optionally substituted carbon chain, or a combination thereof. The optionally substituted carbon chain may incorporate one or more cyclic groups. The one or more cyclic groups may be selected from an optionally substituted C₃₋₁₂ cycloalkylene, an optionally substituted C₆₋₁₀ arylene, an optionally substituted 3 to 12 membered heterocyclene and an optionally substituted 5 to 10 membered heteroarylene.

The cycloalkylene, arylene, heterocyclene or heteroarylene may be unsubstituted or substituted with one or more of oxo, OR⁷, NR⁷R⁸, COOR⁷ OCOR⁷, CONR⁷R⁸ or NR⁷COR⁸, wherein R⁷ and R⁸ are independently H, C₁₋₁₂ alkyl, C₂₋₁₂ alkenyl or C₂₋₁₂ alkynyl.

The or each heteroatom may be selected from the group consisting of NR⁵, O and S, wherein R⁵ is as defined above. R⁵ may be H, optionally substituted C₁₋₆ alkyl, optionally substituted C₂₋₆ alkenyl or optionally substituted C₂₋₆ alkynyl. More preferably, R⁵ is H, optionally substituted C₁₋₃ alkyl, optionally substituted C₂₋₃ alkenyl or optionally substituted C₂₋₃ alkynyl. Accordingly, R⁵ may be H or —CH₂CHCH₂.

The carbon chain maybe unsubstituted or substituted with one or more of oxo, OR⁷, NR⁷R⁸, COOR⁷, OCOR⁷, CONR⁷R⁸, NR⁷COR⁸, optionally substituted alkyl, optionally substituted alkenyl or optionally substituted alkynyl, where R⁷ and R⁸ are as defined above. An optionally substituted alkyl, optionally substituted alkenyl or optionally substituted alkynyl may be unsubstituted or substituted with one or more of oxo, OR⁷, NR⁷R⁸, COOR⁷, OCOR⁷, CONR⁷R⁸ and NR⁷COR⁸.

Preferably, the linker is an optionally substituted carbon chain optionally interrupted by one or more heteroatoms. The carbon chain may incorporate one or more of optionally substituted C₃₋₉ cycloalkylene, optionally substituted C₆₋₁₀ arylene, optionally substituted 3 to 8 membered heterocycylene and/or optionally substituted 5 to 8 membered heteroarylene groups. The carbon chain may be substituted with one or more oxo groups, one or more OH groups and/or one or more methyl groups.

In some embodiments, the optionally substituted C₆₋₁₀ arylene is optionally substituted phenylene.

In some embodiments, the optionally substituted 3 to 8 membered heterocycylene is a 5 or 6 membered heterocycylene. The optionally substituted heterocycylene may be optionally substituted piperidinylene or optionally substituted piperazinylene.

The linker may have a length of between 1 and 50, between 2 and 30, between 2 and 20 atoms, between 3 and 10 atoms, between 3 and 7 atoms or between 4 or 6 atoms. In embodiments where the linker incorporates cyclic groups, the length of the linker may be understood to be counted as the shortest possible length from end to end.

Accordingly, the linker may be

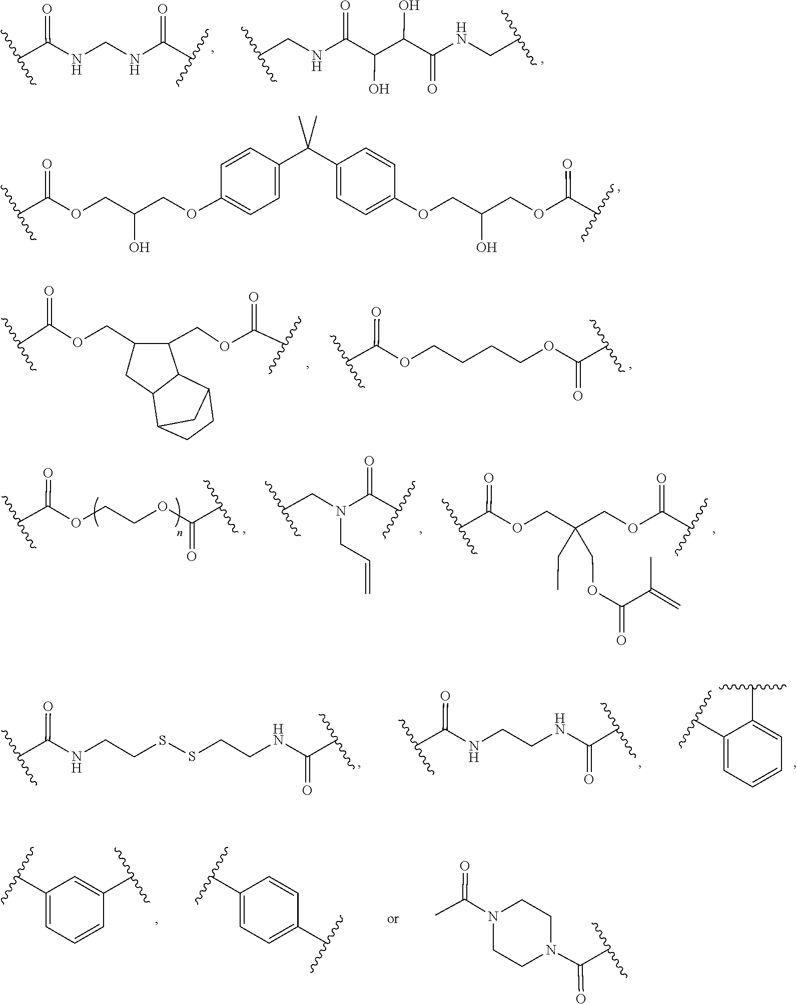

where n is an integer of at least 1. n may be an integer between 1 and 100, between 2 and 50, between 3 and 25 or between 4 and 10.

The crosslinking agent may be N,N-methylene bisacrylamide, N,N′-diallyltartardiamide, 2,2-bis[4-(2-hydroxy-3-methacryloxypropoxy)phenyl]propane, tricyclodecane dimethanol diacrylate, 1,4-butanediol diacrylate, tetraethylene glycol diacrylate, polyethylene glycol dimethacrylate, N,N-diallylacrylamide, trimethylpropane trimethylacrylate, N,N′-bis(acryloyl)cystamine N,N′-bis(acryloyl)ethylenediamine, divinylbenzene or N,N′-bisacryloylpiperazine.

The plurality of monomers may be present in the solution at a concentration between 0.0005 and 1,000 mM, between 0.001 and 500 mM, between 0.005 and 100 mM, between 0.01 and 50 mM, between 0.05 and 25 mM, between 0.1 and 10 mM, or between 0.2 and 5 mM. When the plurality of monomers comprise more than one chemical species, the concentration may be understood to be the combined concentration of the more than one chemical species.

The molar ratio of the one or more crosslinking agents to the rest of the plurality of monomers may be between 1:1 and 1:500, between 1:5 and 1:250, between 1:10 and 1:100, between, 1:20 and 1:75, between 1:25 and 1:50 or between 1:30 and 1:40. The rest of the plurality of monomers may be understood to be all of the plurality of monomers besides from the one or more crosslinking agents.

The method may comprise contacting the catalyst particle, oxidant and plurality of monomers in the presence of a template species, such that the polymeric shell is or comprises a molecular imprinted polymer (MIP).

The template species may be one or more of an ion, a metal complex, an organic compound, a carbohydrate, an oligosaccharide, a polysaccharide, a drug, a toxin, a pesticide, a biomarker, a peptide, a protein, a nucleoprotein, a mucoprotein, a lipoprotein, a synthetic protein, a glycoprotein, a nucleic acid, a biological receptor, a receptor fragment, a cell, a cell fragment, a tissue fragment, a bacterium, a virus, a microorganism, a tissue sample, an inorganic crystal, a protein crystal, a glucosaminoglycan, a steroid, an immunosuppressant, a hormone, a heparin, an antibiotic and/or vitamin. In principle, any small molecule could be a template. For instance, the organic compound may have a molecular weight of at least 20 g/mol, at least 50 g/mol, at least 75 g/mol, at least 100 g/mol, at least 125 g/mol, at least 150 g/mol or at least 175 g/mol. The organic compound may have a molecular weight of less than 1,000 g/mol, less than 500 g/mol, less than 300 g/mol, less than 250 g/mol, less than 200 g/mol, less than 175 g/mol or less than 150 g/mol. The organic compound may have a molecular weight of between 20 and 1,000 g/mol, between 50 and 500 g/mol, between 75 and 300 g/mol, between 100 and 250 g/mol or between 125 and 200 g/mol. For instance, the organic compound may be 4-nitrophenol, 0-nitrophenol, amphetamine, citric acid, hydrocortisone, cocaine, tetrahydrocannabinol (THC) or fentanyl.

The template species may be bound to a substrate. The substrate may be a solid substrate. The substrate may comprise or be a metal or metalloid or an oxide thereof, glass or a polymer. The metal or metalloid maybe a pure metal, a pure metalloid, an impure metal, an impure metalloid, an alloy, a metal containing compound or a metalloid containing compound. Accordingly, the substrate may be or comprise iron oxide. The substrate maybe in the form of a bead. Accordingly, the template species may be bound to a glass bead. It should be appreciated that a plurality of template species may be bound to a plurality of solid substrates. Accordingly, a plurality of template species may be bound to a plurality of beads.

Alternatively, the template species may be dissolved in a solution. Accordingly, in some embodiments, the template species is not bound to a substrate.

The template species may be present in the solution at a concentration between 0.01 and 1,000 mM, between 0.05 and 500 mM, between 0.1 and 100 mM, between 0.5 and 50 mM, between 1 and 25 mM, between 2 and 10 mM or between 4 and 8 mM.

The molar ratio of the plurality of monomers to the template species maybe between 10:1 and 1:10, between 5:1 and 1:5, between 2:1 and 1:3, between 1.5:1 and 1:2 or between 1:1 and 1:1.5.

The method may comprise contacting the catalyst particle, oxidant and plurality of monomers in the presence of a chain transfer agent. The chain transfer agent may be an optionally substituted mercaptan, an alcohol, an amine, a silane, a halogen, a halogenated alkane, an aromatic hydrocarbon or carbon disulfide. The mercaptan may be an optionally substituted C₁₋₂₀ mercaptan, and maybe 2-mercaptoethanol, dodecyl mercaptan, thioglycolic acid or 4-methylbenzenethiol. The alcohol maybe a C₁₋₂₀ alcohol, a C₂₋₁₀ alcohol or a C₃₋₈ alcohol, and may be isopropanol or benzyl alcohol. The amine may be a C₁₋₁₀ or C₃₋₈ amine, and may be as triethylamine or diisopropylamine. The silane may be a C₁₋₁₀ or C₂₋ₛ silane and may be trimethylsilane or triethylsilane. The halogen may be chlorine or bromine. The halogenated alkane may be a C₁₋₁₀, a C₁₋₅ or a C₁₋₂ halogenated alkane, and may be carbon tetrachloride. The aromatic hydrocarbon maybe pentaphenylethane.

The molar ratio of the plurality of monomers to the chain transfer agent may be between 10,000:1 and 1:1, between 5,000:1 and 2:1, between 2,500:1 and 5:1 or between 1,000:1 and 10:1.

It maybe appreciated that in some embodiments, the present invention describes the application of living polymerization for producing soluble or colloidal MIP particles. It may be appreciated that any known living free-radical polymerization techniques may be applied to the method of the first aspect, such as iniferter polymerization, nitroxide-mediated radical polymerization, atom-transfer radical polymerization (ATRP) and reversible addition-fragmentation chain-transfer (RAFT) polymerization. These open new routes for the synthesis of polymers with controlled and relatively low-molecular weights. Controlled/living polymerization techniques are based on a delicate balance between dormant and active species that effectively reduces the concentration of free radicals in the system and minimizes the extent of termination. Living polymerization could be free of side reactions such as termination and chain transfer and thus can generate polymers with well-defined molecular weight distribution and structure. The same approach can be applied to copolymers, thus making it possible to produce block copolymers by free radical polymerisation by proper sequencing of the monomer additions.

Typically the reaction is stopped at an early stage producing polymers with molecular weight 500-1,000,000 Da. It may be appreciated that this is intrinsic property of living polymerisation. Other ways to stop the reaction at an early stage may include cancelling irradiation in case of light triggered reaction, or flushing oxygen in case of chemical initiation.

The method may be conducted at a temperature between −25 and 1000° C., between 0 and 800° C., between 5 and 600° C., between 1° and 40° C., between 15 and 30° C. or between 2° and 25° C. Advantageously, the method maybe conducted at room temperature.

The method may comprise contacting the catalyst particle, the oxidant and the plurality of monomers for between 1 minutes and 72 hours, between 15 minutes and 48 hours, between 30 minutes and 24 hours, between 45 minutes and 12 hours, between 1 and 6 hours, between 2 and 4 hours or between 2.5 and 3.5 hours. It may be appreciated that in some embodiments, the method may comprise contacting the catalyst particle precursor, the oxidant and the plurality of monomers for between 1 minutes and 72 hours, between 15 minutes and 48 hours, between 30 minutes and 24 hours, between 45 minutes and 12 hours, between 1 and 6 hours, between 2 and 4 hours or between 2.5 and 3.5 hours.

The method may be conducted in a batch process or in a continuous process.

Accordingly, the method may be conducted in a microfluidic reactor or in a flow reactor.

The method may comprise contacting the catalyst particle, the oxidant and the plurality of monomers under UV light. The catalyst particle, the oxidant and the plurality of monomers may be contacted under UV light for at least 1 second, at least 15 seconds, at least 30 seconds, at least 45 seconds or at least 1 minute. The catalyst particle, the oxidant and the plurality of monomers may be contacted under UV light for between 15 seconds and 30 minutes, between 30 seconds and 10 minutes, between 45 seconds and 5 minutes, or between 1 and 3 minutes. It may be appreciated that contacting the catalyst particle, the oxidant and the plurality of monomers under UV light may initiate the reaction.

The method may comprise isolating the composite particle.

The composite particle may be isolated from one or more unreacted low molar mass components and/or one or more by-products of the reaction. The composite particle may be isolated by affinity or magnetic separation, dialysis, extraction, filtration or centrifugation. The filtration maybe ultrafiltration or nanofiltration. The composite particle maybe isolated from the solid phase. Accordingly, the composite particle may be isolated by solid phase extraction.

The method may comprise conjugating the composite particle to a further species. This may be conducted after isolating the composite particle. The further species may be as defined above. The method may comprise conjugating the polymeric shell to the further species. Advantageously, the resultant nanocomposites maybe used in diagnostics and medicine.

It is believed that composite particles produced using the method of the first aspect are novel and inventive per se.

Accordingly, in a second aspect, there is provided a composite particle obtained or obtainable by the method of the first aspect.

The composite particles produced in the method of the first aspect maybe used in a variety of applications. It is believed these applications are novel and inventive per se.

Accordingly, in accordance with a third aspect there is a provided use of a composite particle in sensing a target molecule, wherein the composite particle comprises a polymeric shell around a catalyst particle.

Preferably, the use is a use of the composite particle in sensing the concentration of the target molecule in a solution.

The composite particle may be used to sense a target molecule using surface plasmon resonance (SPR), localised surface plasmon resonance (LSPR), optical density, electrochemical methods such voltammetry, amperometry or coulometry, or impedance, resistance measurements, a lateral flow sensor or in a homogeneous assay.

In accordance with a fourth aspect, there is provided a method of sensing a target molecule in a solution, the method comprising:
contacting a composite particle with a solution, wherein the composite particle comprises a polymeric shell around a catalyst particle; and
sensing an optical property of the catalyst particle, and thereby sensing the target molecule in the solution.

Preferably, the composite particle is produced using the method of the first aspect or is the composite particle of the second aspect.

Preferably, the composite particle is as described above. Preferably, the polymeric shell comprises a molecularly imprinted polymer (MIP). Preferably, the MIP is imprinted with the target molecule.

The optical property may be surface plasmon resonance (SPR) absorbance, localised surface plasmon resonance (LSPR) absorbance or fluorescence. Preferably, the method comprises determining a value for the optical property.

Preferably, the method comprises calculating the concentration of the target molecule in the solution. The concentration of the target molecule in the solution may be calculated by comparing the value of the optical property of the composite particle in the solution to a calibration curve including values of the optical property for different known concentrations of the target molecule in solution, and thereby calculating the concentration of the target molecule in the solution.

The target molecule may be one or more of an ion, a metal complex, an organic compound, a drug, a toxin, a pesticide, a biomarker, a peptide, a protein, a receptor, a receptor fragment, a cell, a cell fragment, a tissue fragment, a bacterium, a virus, an inorganic crystal and/or a protein crystal. The organic compound may be the same as defined above in relation to the template species. The organic compound may be 4-nitrophenol, 0-nitrophenol, pentaerythritol tetranitrate, amphetamine, citric acid, hydrocortisone, cocaine or fentanyl.

In accordance with a fifth aspect, there is provided a method of sensing a target molecule in a solution, the method comprising:
disposing an electrode with a composite particle immobilised thereon, and a further electrode in a solution, wherein the composite particle comprises a polymeric shell around a catalyst particle;
applying a voltage across the electrodes; and measuring the current, to thereby sensing the target molecule in the solution.

Preferably, the composite particle is produced using the method of the first aspect or is the composite particle of the second aspect.

Preferably, the composite particle is as described above. Preferably, the polymeric shell comprises a molecularly imprinted polymer (MIP). Preferably, the MIP is imprinted with the target molecule.

The target molecule may be one or more of an ion, a metal complex, an organic compound, a drug, a toxin, a pesticide, a biomarker, a peptide, a protein, a receptor, a receptor fragment, a cell, a cell fragment, a tissue fragment, a bacterium, a virus, an inorganic crystal and/or a protein crystal. The organic compound may be the same as defined above in relation to the template species.

The method may comprise immobilising the composite particle on the electrode. Immobilising the composite particle on the electrode may comprise covalently immobilising the composite particle on the electrode. Immobilization can be conducted using techniques known in the art, such as an EDC/NHS (1-ethyl-3-(3-dimethylaminopropyl)carbodiimide/N-hydroxysuccinimide) activation mechanism, using mercaptan compounds or a silane compound comprising amino or carboxylic groups.

Preferably, applying the voltage across the two electrodes and measuring the current comprises applying differential pulse voltammetry (DPV). Accordingly, the method may comprise applying a first pulse with a base potential across the electrodes and applying further pulses across the electrodes, such that the potential increases with each pulse. Preferably, the potential increases by the same amount with each pulse, i.e. the potential increases with a linear ramp. Preferably, the current is measured before the pulse application and at the end of the pulse, and the difference between them is recorded.

Preferably, the method comprises calculating the concentration of the target molecule in the solution. The concentration of the target molecule in the solution may be calculated by comparing the current response at a given voltage to a calibration curve of the current response at the given voltage when the electrode is disposed in solution comprising different known concentrations of the target molecule, and thereby calculating the concentration of the target molecule in the solution.

The target molecule may be as defined above.

In a sixth aspect, there is a provided use of a composite particle in a lateral flow sensor, wherein the composite particle comprises a polymeric shell around a catalyst particle.

In a seventh aspect there is provided a lateral flow sensor comprising a composite particle, the composite particle comprising a polymeric shell around a catalyst particle.

It may be appreciated that a lateral flow sensor could also be known as a lateral flow test (LFT), a lateral flow device (LFD) or a lateral flow immunochromatographic assay.

The lateral flow sensor may comprise a sandwich assay or a competitive assay.

Preferably, the composite particle is produced using the method of the first aspect or is the composite particle of the second aspect.

Preferably, the composite particle is as described above. The polymeric shell may comprise a molecularly imprinted polymer (MIP). Preferably, the MIP is imprinted with a target molecule.

The target molecule may be one or more of an ion, a metal complex, an organic compound, a drug, a toxin, a pesticide, a biomarker, a peptide, a protein, a receptor, a receptor fragment, a cell, a cell fragment, a tissue fragment, a bacterium, a virus, an inorganic crystal and/or a protein crystal. The organic compound may be the same as defined above in relation to the template species. The target molecule may be a protein, a biomarker, a drug, a toxin or a hormone. The drug may be a drug of abuse.

Alternatively, the polymeric shell may have a further species attached thereto. The further species may have fluorescent functionality, and/or may be a functional group capable of reducing non-specific binding (e.g. PEG).

The lateral flow sensor may comprise a sample receiving section, configured to receive a sample to be tested and a test section comprising one or more test lines, and optionally also a control line, and the lateral flow sensor may be configured to allow the sample to flow from the sample receiving section to the test section. The sample may flow by capillary flow.

The sample may be understood to be a liquid sample. The sample may comprise or be a bodily fluid.

The sample receiving section may comprise a sample pad. The sample pad maybe configured to act as a filter to aid flow of the sample. The sample pad may be treated to adjust a property of the sample, e.g. pH or viscosity.

The lateral flow sensor may comprise a conjugate section disposed between the sample receiving section and the test section test section. The conjugate section may comprise a conjugate release pad. The conjugate section preferably comprises one or more conjugates. Preferably, the conjugate release pad is configured to release the one or more conjugates such that they flow to the test section with the sample. The one or more conjugates may comprise one or more conjugates configured to bind to one or more target molecules in the sample. Alternatively, or additionally, the one or more conjugates may comprise one or more conjugates configured to bind to the control line. The one or more conjugates may comprise one or more antibodies, one or more MIPs, one or more aptamers and/or the composite particle. The one or more conjugates may each be configured to bind to a target molecule and/or an antibody.

The test section may comprise a membrane. The membrane may be a nitrocellulose membrane.

In one embodiment, the one or more test lines are configured to capture one or more target molecules. It may be appreciated that this is a sandwich assay. Preferably, the one or more test lines comprise immobilised molecules configured to conjugate with the one or more target molecules, wherein the one or more immobilised molecules define a line. Preferably, the one or more immobilised molecules define a line across the test section. Preferably, the line is substantially perpendicular to the direction of flow of the sample. The one or more immobilised molecules maybe or comprise antibodies and/or the composite particle.

In an alternative embodiment, the one or more test lines comprise immobilised molecules, wherein the immobilised molecules are the one or more target molecules or an analogue thereof. It may be appreciated that this is a competitive assay. Preferably, the one or more immobilised molecules define a line across the test section. Preferably, the line is substantially perpendicular to the direction of flow of the sample.

The control line maybe configured to capture the one or more conjugates configured to bind thereto. Preferably, the control line comprises immobilised molecules configured to conjugate with the one or more conjugates configured to bind to the control line. Preferably, the one or more immobilised molecules define a line across the test section. Preferably, the line is substantially perpendicular to the direction of flow of the sample. The one or more immobilised molecules may be or comprise antibodies and/or the composite particle.

The lateral flow sensor may comprise a wicking pad. The wicking pad may be disposed adjacent to the test section. The test section may be disposed between the sample receiving section and the wicking pad. The test section may be disposed between the conjugate section and the wicking pad. Accordingly, the lateral flow sensor may define a strip, the sample receiving section maybe disposed substantially adjacent a first end of the strip and the wicking pad may be disposed substantially adjacent to a second end of the strip, wherein the second end is opposite the first end. The lateral flow sensor may be configured to enable the sample to flow in a direction substantially from the first end to the second end.

The wicking pad is preferably configured to absorb fluid. The wicking pad preferably comprises a porous material.

In accordance with an eighth aspect, there is provided a method of sensing a target molecule in a sample, the method comprising contacting the sample with the lateral flow sensor of the seventh aspect and observing whether or not a test line appears on the lateral flow sensor, and thereby sensing the target molecule in a sample.

Advantageously, the composite particles of the invention can be used in a diagnostic test.

In a ninth aspect, there is provided a method of sensing a target molecule in a sample, the method comprising:
contacting a spectrophotometric reagent, a composite particle, an oxidising or reducing agent and the sample, wherein the composite particle comprises a polymeric shell around a catalyst particle; and
measuring the absorbance of the resultant composition, to thereby sensing the target molecule in the sample.

Preferably, the composite particle is produced using the method of the first aspect or is the composite particle of the second aspect.

Preferably, the polymeric shell comprises a molecularly imprinted polymer (MIP). Preferably, the MIP is imprinted with the target molecule.

The target molecule may be one or more of an ion, a metal complex, an organic compound, a drug, a toxin, a pesticide, a biomarker, a peptide, a protein, a receptor, a receptor fragment, a cell, a cell fragment, a tissue fragment, a bacterium, a virus, an inorganic crystal and/or a protein crystal. The organic compound may be the same as defined above in relation to the template species.

Preferably, the catalyst particle comprises or consists of a material which can catalyse a bleaching or colour enhancing reaction between the spectrophotometric reagent and the oxidising or reducing agent. Accordingly, the catalyst particle may comprise or consist of gold, silver or a magnetic material, like iron oxide.

It may be appreciated that the above method may be described as a homogeneous assay.

The spectrophotometric reagent may be a redox sensitive dye. In some embodiments, the spectrophotometric reagent may be bromopyrogallol red, 3,3′,5,5′-tetramethylbenzidine (TMB) or 2,2′-azino-bis(3-ethylbenzothiazoline-6-sulfonic acid) (ABTS).

The oxidising agent may be a peroxide, a persulfate or ozone. The peroxide may be hydrogen peroxide. The persulfate might be ammonium persulfate.

The sample may comprise a buffer. The buffer may be a phosphate, acetate, or borate buffer.

The method may comprise contacting the spectrophotometric reagent, the oxidising or reducing agent, the composite particle and the sample for a predetermined time prior to measuring the absorbance of the resultant composition. The predetermined time may be determined by the skilled person. For instance, the predetermined time may be between 15 seconds and 24 hours, between 30 seconds and 12 hours, between 1 minute and 6 hours, between 5 minutes and 2 hours, between 10 and 90 minutes, between 15 and 60 minutes, between 20 and 45 minutes, between 25 and 35 minutes or between 28 and 32 minutes.

Preferably, the method comprises calculating the concentration of the target molecule in the sample. The concentration of the target molecule in the sample may be calculated by comparing the measured absorbance of the resultant composition to a calibration curve of the absorbance for compositions comprising reference samples with known concentrations of the target molecule, and thereby calculating the concentration of the target molecule in the samples.

In a tenth aspect, there is provided use of a composite particle in catalysis, wherein the composite particle comprises a polymeric shell around a catalyst particle.

The composite particle is preferably produced using the method of the first aspect or is the composite particle of the second aspect.

The use maybe in catalysing an oxidative or a hydrolytic reaction. They can also be used to catalyse reactions in diagnostic assays, such as the conversion of a non-fluorescent molecule into a fluorescent molecule which can be detected using a fluorescence reader.

Advantageously, the polymer coating can assist with enhancing specificity of the reaction by restricting access to the catalytic sites for particular compounds of appropriate size and charge. Alternatively, polymer coatings can assist in preconcentration of the substrate leading to increasing yield of catalytic reaction.

In an eleventh aspect, there is provided a pharmaceutical composition comprising a composite particle and a pharmaceutically acceptable carrier, wherein the composite particle comprises a polymeric shell around a catalyst particle

In an eleventh aspect, there is provided a composite particle or the pharmaceutical composition of the eleventh aspect for use in a method of treatment or diagnosis, wherein the composite particle comprises a polymeric shell around a catalyst particle.

In a twelfth aspect, there is provided a composite particle or the pharmaceutical composition of the eleventh aspect for use in a method of imaging, wherein the composite particle comprises a polymeric shell around a catalyst particle.

The composite particle is preferably produced using the method of the first aspect or is the composite particle of the second aspect.

The composite particle may comprise an antibody or an aptamer conjugated to the polymeric shell. Alternatively, or additionally, the polymeric shell may comprise an MIP. The antibody, aptamer or MIP may be configured to deliver the composite particle to a specific target. Preferably, the specific target is in the body of a patient who has been administered the composite particle. The target maybe a membrane protein or a cell.

Advantageously, a composite particle with a high contrast ratio can be delivered specifically to a target in a patient's body due to the antibody, aptamer or MIP. For instance, a composite particle with an iron oxide core maybe understood to have a high contrast ratio.

In a thirteenth aspect, there is provided a composite particle or the pharmaceutical composition of the eleventh aspect for use in drug delivery, wherein the composite particle comprises a polymeric shell around a catalyst particle.

The composite particle is preferably produced using the method of the first aspect or is the composite particle of the second aspect.

The composite particle may comprise an antibody or an aptamer conjugated to the polymeric shell. Alternatively, or additionally, the polymeric shell may comprise an MIP. The antibody, aptamer or MIP may be configured to deliver the composite particle to a specific target. Preferably, the specific target is in the body of a patient who has been administered the composite particle. The target may be a membrane protein or a cell.

The composite particle may comprise a drug molecule adsorbed therein.

The catalyst particle may comprise a magnetic material, such as iron oxide.

Advantageously, after the composite particles have been delivered to a patient, the composite particle can be heated in magnetic field providing release of the adsorbed drug molecule adjacent to the target.

In accordance with a fourteenth aspect there is provided a sensor comprising:
a substrate;
a plurality of nanoparticles disposed on the substrate; and
a plurality of composite particles disposed on the substrate, wherein the composite particles comprise a polymeric shell around a catalyst particle.

Preferably, the plurality of composite particles are produced using the method of the first aspect or are the composite particles of the second aspect. Preferably, the composite particles are as described above. Preferably, the composite particles are disposed in a layer over a surface of the substrate. Preferably, the composite particles are disposed in a layer over a surface of the substrate and over the nanoparticles.

Preferably, the polymeric shell is as defined above. Preferably, the polymeric shell comprises a molecularly imprinted polymer (MIP). Preferably, the MIP is imprinted with a target molecule.

Preferably, the substrate comprises or consists of a material which allows the transmission of light therethrough. Preferably, the substrate comprises or consists of a material which allows the transmission of visible light therethrough. For example, the substrate may comprise or be glass or a polymer.

The plurality of nanoparticles are preferably a plurality of metal nanoparticle, and more preferably a plurality of silver and/or gold nanoparticles.

Preferably, the plurality of nanoparticles are randomly distributed. Preferably the nanochip nanoparticles are arranged such a way that allows surface plasmon resonance (SPR) or localised surface plasmon resonance (LSPR).

In accordance with a fifteenth aspect, there is provided a method of sensing a target molecule in a fluid, the method comprising:
providing the sensor of the fourteenth aspect;
contacting the sensor with a fluid; and
sensing an optical property of the sensor, and thereby sensing the target molecule in the fluid.

The fluid may be or comprise a liquid or a gas. The liquid may be or comprise a solution or a suspension. In some embodiments, the fluid is a gas. The gas may comprise air.

The optical property may be surface plasmon resonance (SPR) absorbance, localised surface plasmon resonance (LSPR) absorbance or fluorescence. Preferably, the optical property is localised surface plasmon resonance (LSPR) absorbance. Accordingly, the optical property of the substrate may be determined using a photometer.

Preferably, the method comprises determining a value for the optical property of the sensor. Determining a value for the optical property may comprise removal of background optical signal. Removal of background optical signal may comprise removal of the optical signal generated by a reference sensor, wherein the reference sensor is contacted with a reference fluid, wherein the reference does not comprise the target molecule. Aside from omission of the target molecule the reference fluid may be the same as the fluid which comprises the target molecule.

Preferably, the method comprises calculating the amount or concentration of the target molecule in the fluid. The amount of the target molecule in the fluid may be calculated by comparing the value of the optical property of the substrate in contact with the fluid to a calibration curve including values of the optical property for different known amounts or concentrations of the target molecule in the fluid, and thereby calculating the amount of the target molecule in the fluid.

The target molecule may be as described in the fourth aspect.

In a sixteenth aspect there is provided a sensor comprising
an optical fibre extending between a first end and a second end, the optical fibre comprising a sensing portion, wherein the sensing portion is configured to allow generation of an evanescent field; and
a composite particle disposed on the sensing portion of the optical fibre, the composite particle comprising a polymeric shell around a catalyst particle.

Preferably, the composite particle is produced using the method of the first aspect or is the composite particle of the second aspect.

Preferably, the composite particle is as described above. Preferably, the polymeric shell comprises a molecularly imprinted polymer (MIP). Preferably, the MIP is imprinted with the target molecule.

The optical fibre may comprise a core extending between the first end and the second end. The core may comprise or consist of a plastic, glass or a combination thereof. The plastic and/or glass maybe doped.

The optical fibre may further comprise a cladding layer disposed circumferentially around the core. The cladding layer may extend between the first end and the second end. The cladding layer may comprise or consist of a plastic, glass or a combination thereof.

Preferably, the index of refraction of the core is greater than the index of refraction of the cladding layer.

The optical fibre may further comprise a buffer layer disposed circumferentially around the cladding layer.

It may be understood that an optical signal may propagate through the optical fibre within the core. It may be understood that the sensing portion of the optical fibre is configured such that an evanescent field is generated on the surface of the sensing portion when an optical signal is propagated through the optical fibre.

In some embodiments the sensing portion comprises a tapered portion of the optical fibre. In particular, the tapered portion would be understood to have a smaller diameter compared to an adjacent portion of the optical fibre.

Accordingly, the tapered potion of the optical fibre may comprise a cladding layer with a reduced thickness compared to the adjacent portion. Alternatively, the sensing portion of the optical fibre may comprise no cladding layer.

The tapered portion maybe disposed adjacent the first end of the optical fibre. The adjacent portion may extend between the tapered portion and the second end of the optical fibre. Alternatively, the tapered portion may be disposed adjacent the second end of the optical fibre. The adjacent portion may extend between the first end of the optical fibre and the tapered portion.

In some embodiments, the optical fibre comprises a tapered region disposed between and spaced apart from the first and second ends. The optical fibre may comprise a first adjacent portion which extends between the first end of the optical fibre and the tapered portion. The optical fibre may comprise a second adjacent portion which extends between the tapered portion and the second end of the optical fibre. The first and second adjacent portions may have substantially the same diameter as each other.

Preferably, the sensor further comprises a light source capable of emitting an optical signal. The light source may comprise or be an LED (light emitting diode) and/or a laser. The light source may be configured to provide an optical signal to the optical fibre. The light source may be configured to input the optical signal into the first end of the optical fibre. The light source may be connected to the first end of the optical fibre. Accordingly, the first end of the optical fibre maybe referred to as the input end of the optical fibre.

Preferably the sensor further comprises a detector capable of detecting an optical signal. The detector may be an optical spectrometer or a photodiode. The detector may be configured to receive and detect an optical signal from the optical fibre, and preferably from the second end thereof. The detector may be connected to the second end of the optical fibre. The second end of the optical fibre may be referred to as the output end of the optical fibre.

Accordingly, the light source, detector and optical fibre may be configured such that an optical signal can be generated by the light source, propagated along the optical fibre and detected by the detector.

The sensor may further comprise a processor configured to process the data generated by the detector.

In accordance with a seventeenth aspect, there is provided a method of sensing a target molecule in a fluid, the method comprising:
providing the sensor of the sixteenth aspect;
contacting the composite particle with the fluid;
inputting an optical signal into the first end of the optical fibre, and thereby generating an evanescent field adjacent the sensing portion of the optical fibre, such that the composite particle is at least partially disposed in the evanescent field; and
detecting an output optical signal or value from the second end of the optical fibre and comparing the output optical signal to a reference optical signal or value to thereby sense the target molecule in the fluid.

The fluid may be defined as described in the fifteenth aspect.

Preferably, the composite particle is produced using the method of the first aspect or is the composite particle of the second aspect.

Preferably, the composite particle is as described above. Preferably, the polymeric shell comprises a molecularly imprinted polymer (MIP). Preferably, the MIP is imprinted with the target molecule.

Detecting an output optical value may comprise detecting an intensity of the optical signal at a given a wavelength.

The reference signal may be an optical signal which is detected from the second end of the optical fibre in the absence of the target molecule. The reference value may be the intensity of an optical signal at a given wavelength detected from the second end of the optical fibre in the absence of the target molecule.

The method may comprise calculating the amount or concentration of the target molecule in the fluid. The amount or concentration of the target molecule in the fluid may be calculated by comparing the output optical signal or value with further reference signals or values wherein the further reference signals or values are the optical signals which are detected from the second end of the optical fibre in the presence of different known amounts or concentrations of the target molecule. The further reference values may be the intensity of the optical signal at the given wavelength detected from the second end of the optical fibre in the presence of different known amounts or concentrations of the target molecule. Accordingly, the method may comprise comparing the output optical value to a calibration curve of reference values for different known amounts of the target molecule in the fluid, and thereby calculating the amount or concentration of the target molecule in the fluid.

The target molecule may be as described in the fourth aspect.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying drawings, in which:

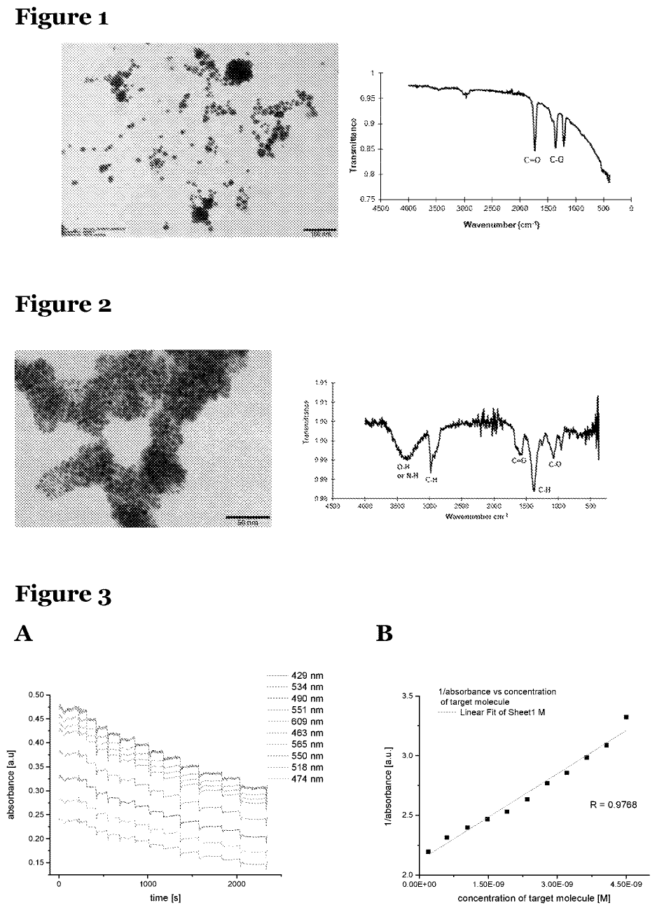

FIG. 1 shows a transmission electron microscopy (TEM) image and a Fourier-transform infrared (FTIR) spectra of gold nanoparticles coated with polyacrylic acid;

FIG. 2 TEM image and FTIR spectrum of gold nanoparticles coated with molecular imprinted polymers (MIPs);

FIG. 3 shows (A) the localized surface plasmon resonance (LSPR) signal monitored over time with increasing concentrations of an analyte present in a solution comprising gold nanoparticles coating with MIPs; and (B) a calibration plot showing the absorbance against concentration;

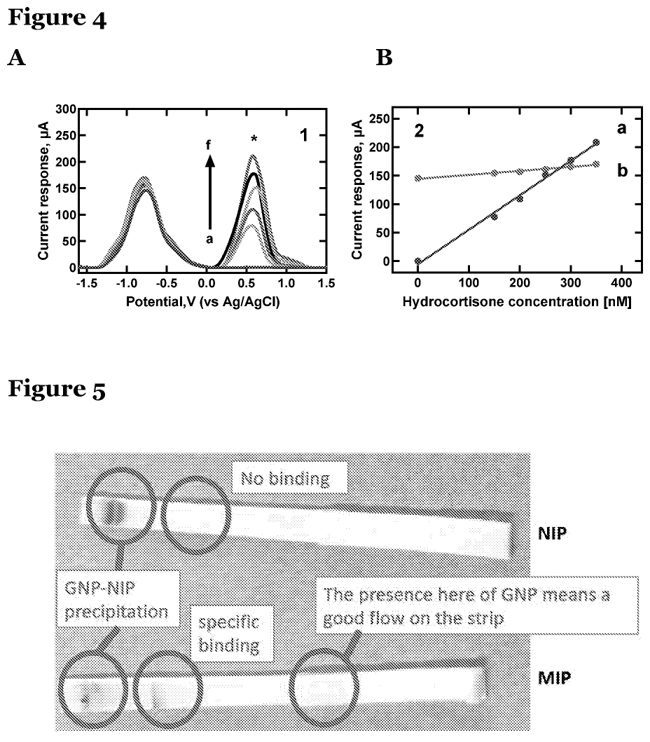

FIG. 4 shows (A) the differential pulse voltammetry (DPV) response of a sensor based on gold modified with nanoMIP to hydrocortisone standard solutions at (a) 0, (b) 150, (c) 200, (d) 250, (e) 300, and (f) 350 nM in 5 mM PBS; and (B) the calibration plot of (a) the response of the sensor comprising the gold nanoparticle modified with nanoMIP to the hydrocortisone solutions; and (b) the response of a sensor containing non-functionalized gold nanoparticles to the hydrocortisone solutions;

FIG. 5 shows a lateral flow device based on pad adsorbent with control line of hydrocortisone-bovine serum albumin (BSA);

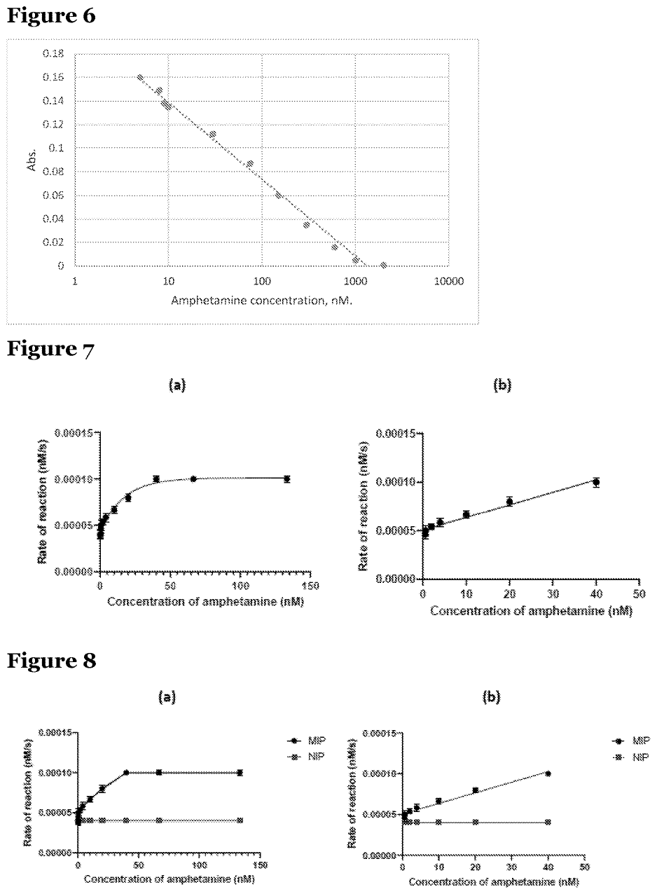

FIG. 6 is a calibration plot showing bromopyrogallol red (BPR) absorbance against concentration of amphetamine;

FIG. 7 shows plots of BRP assay responses (BRP rate of reaction) (A) with amphetamine concentration in the range 0-133 nM and (B) with amphetamine concentration in the range 0-40 nM;

FIG. 8 shows plots of BRP assay response to amphetamine using Au/MIP and Au/NIP nanozymes in (A) amphetamine concentration range 0-133 nM and (B) amphetamine concentration range 0-40 nM;

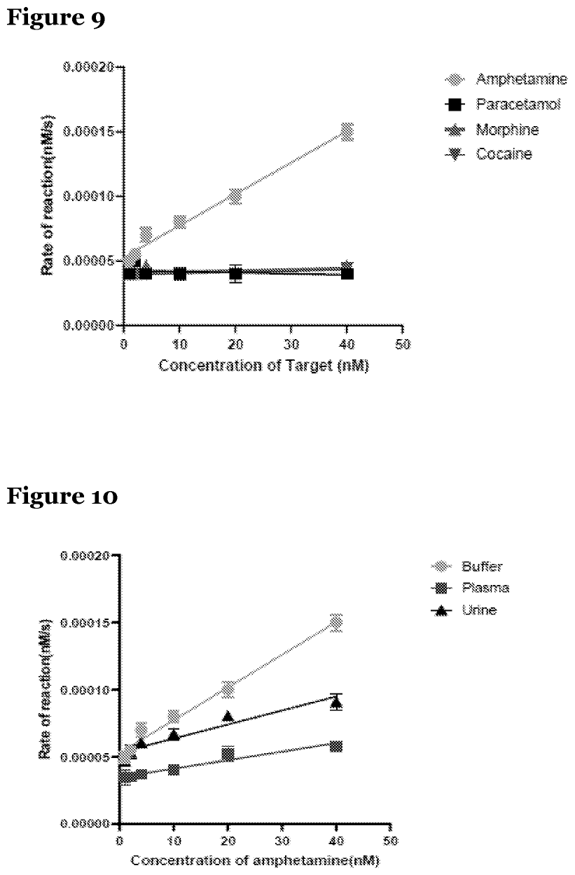

FIG. 9 shows plots of BRP assay responses to 0.3 to 40 nM of amphetamine, paracetamol, morphine and cocaine.;

FIG. 10 shows plots of BRP assay responses to amphetamine in buffer (5 mM PBS, pH 7.0), urine and plasma;

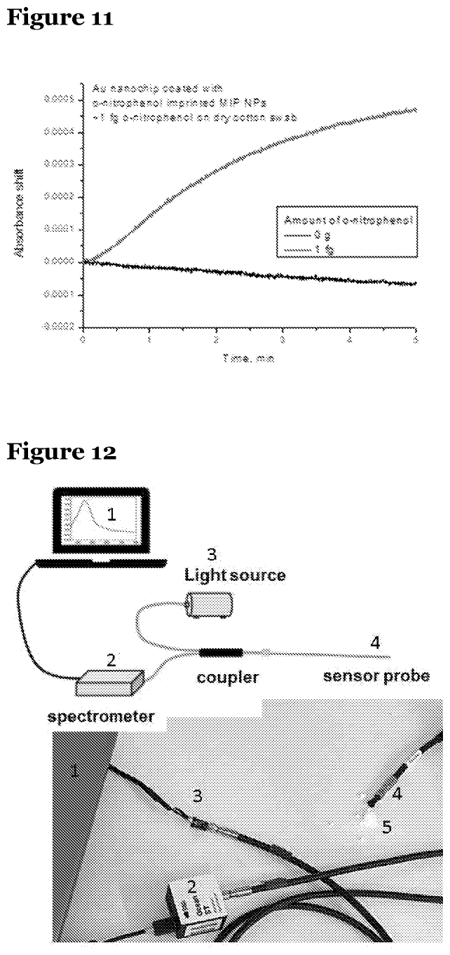

FIG. 11 shows kinetic dependences of absorbance for an LSPR Au nanochip coated with composite gold 0-nitrophenol imprinted MIP nanoparticles upon exposure to air (0 g 0-nitrophenol) and to 0-nitrophenol vapor from a dry cotton swab containing ~1 femtogram of 0-nitrophenol;

FIG. 12 shows an optical-fibre based sensor with a sensor probe comprising composite nanoparticles;

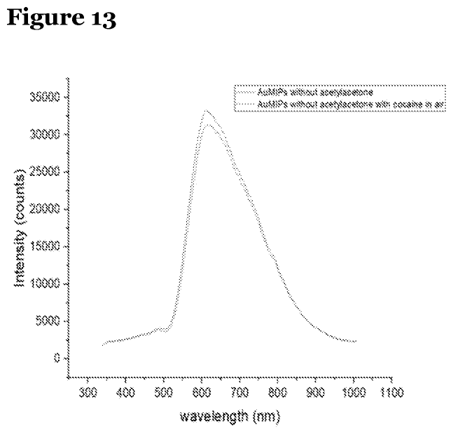

FIG. 13 shows an optical-fibre based sensor response plot upon contact with a target substance (cocaine).

### EXAMPLES

### Example 1. Preparation of Gold Nanoparticles Coated with Polyacrylic Acid

30 μL of precursor solution (6.6 mg mL⁻¹ of hydrogen tetrachloroaurate (III) trihydrate) was added to 10 mL of 10 mM Na-phosphate buffer pH 7.5 and mixed with 0.22 μL of acrylic acid, 500 μL of cyclohexanone and 300 μL of 50 wt % stock solution of hydrogen peroxide. The mixture was incubated for 3 h and nanoparticles purified by dialysis. The nanoparticles were characterized by TEM and IR spectroscopy (FIG. 1). It is noted that in the TEM image it is possible to see a dark core of inorganic material and a light shell of polymer coating.

### Example 2. Preparation of Gold Nanoparticles Covered with Molecularly Imprinted Polymer (MIP)

9 mg of template amphetamine, 3.8 mg of N-isopropylacrylamide, 3.3 mg of N-tert-butylacrylamide, 0.58 mg of N-3-aminopropyl methacrylamide hydrochloride, 0.22 μL of acrylic acid and 0.2 mg of N,N-methylene bisacrylamide were dissolved in 10 mL of 10 mM Na-phosphate buffer, pH 7.5, and mixed with 30 μL of precursor solution (6.6 mg mL⁻¹ of hydrogen tetrachloroaurate (III) trihydrate), 500 μL of cyclohexanone and 300 μL of commercial 50% stock solution of hydrogen peroxide. The mixture was incubated for 3 h and nanoparticles purified by dialysis. The nanoparticles were characterized by EM and IR spectroscopy (FIG. 2). Again, in the TEM image it is possible to see a dark core of inorganic material and a light shell of polymer coating.

### Example 3. Detection of Citric Acid as Explosive Analogue

Gold nanoparticles coated with MIP shell imprinted with citric acid were prepared using the method described in Example 2, but replacing the amphetamine with citric acid. It is noted that citric acid is an analogue for pentaerythritol tetranitrate.

The nanoparticles were mixed in an optical cell with increased concentrations of the target molecules (citric acid). MIPs imprinted for citric acid displayed linear decrease in localized surface plasmon resonance (LSPR) absorbance (FIG. 3 panel a) and these data were used to create a calibration plot (FIG. 3 panel b) allowing a user to calculate the concentration of an unknown solution.

Accordingly, the inventors have demonstrated that the nanoparticles of the invention can be used to determine the concentration of a target molecule in a solution.

### Example 4: Use of Composite Gold-MIP Nanoparticles for Detection of Hydrocortisone

Composite nanoparticles were synthesized with a gold core and MIP shell imprinted with hydrocortisone.

3.8 mg of N-isopropylacrylamide, 3.3 mg of N-tert-butylacrylamide, 0.58 mg of N-3-aminopropyl methacrylamide hydrochloride, 0.22 μL of acrylic acid and 0.2 mg of N,N-methylene bisacrylamide were dissolved in 5 mL of water, then 30 μL of precursor solution (6.6 mg mL⁻¹ of hydrogen tetrachloroaurate (III) trihydrate) was added followed by 300 μL of commercial 50% stock solution of hydrogen peroxide and 500 μL of acetone/acetylacetone mixture in a 2:1 volume ratio. As a control, nanoparticles with a gold core and non-imprinted polymer (NIP) shell were produced in an analogous manner, but without a template.

Nanoparticles were covalently immobilized onto graphite electrodes to produce two sensors, one comprising the gold-MIP nanoparticles and one comprising gold-NIP nanoparticles. The DPV response of the sensor comprising the gold-MIP nanoparticles was measured at 0.59 V (vs Ag/AgCl) as shown in FIG. 4A.

As shown in FIG. 4B, the sensor comprising gold-MIP nanoparticles (represented by line (a)) was significantly more sensitive to changes in the hydrocortisone concentration that the sensor comprising gold-NIP nanoparticles (represented byline (b)).

### Example 5: Application of Composite Nanoparticles in Lateral Flow Sensors

For this test, gold coated with non-imprinted polymer and gold coated with polymer imprinted with hydrocortisone were tested in lateral flow format using strip with a control line of hydrocortisone-bovine serum albumin (BSA). The strip on the top shows elution of the gold-NIP without binding in the control line, whereas, the gold-MIP produced a red coloured control line (FIG. 5, bottom part).

### Example 6. Homogeneous Assay Based on Composite Nanoparticles

An assay was prepared by providing a mixture of bromopyrogallol red (BPR), the gold-MIP nanoparticles of Example 2 in phosphate buffered saline (PBS) and hydrogen peroxide in a well. 200 μL of gold-MIP nanoparticles in PBS, 100 μL of H₂O₂ and 200 μL of bromopyrogallol red solution were used. Samples to be analyzed, in this case solutions of amphetamine with a concentration range of 9 to 1000 nM, were provided in 10 mM PBS. Samples were added to separate wells and allowed to incubate for 30 minutes. The absorbance was then measured, and the results are shown in FIG. 6.

The variance in absorbance is because the MIP-coated gold nanoparticles act as an artificial enzyme, a nanozyme, in the colorimetric oxidation of the BPR dye by peroxide. The mechanism underlying these nanozymes is rooted in the Fenton-like reaction, and involves two key components. First, the gold cores of the nanozyme catalyse the decomposition of hydrogen peroxide (H₂O₂) into hydroxyl radicals (HO·) onto their surfaces that can be detected via colorimetric reactions. Second, the analyte binding to the MIP triggers structural changes in the polymer, mimicking the “induced fit” actions characteristic of enzymes and natural receptors. Specifically, this involves swelling of the nanozyme shell, which exposes sections of the gold core available for catalysis. Consequently, when a target such as amphetamine binds to the MIP, the latter undergoes conformational change, affecting diffusion of the substrate to the catalytic gold surface, and therefore increasing the rate of the BPR oxidation. Therefore, the absorbance of BPR is directly related to the presence of the target (e.g. amphetamine) in solution.

### Example 7. Homogeneous Assay Based on Composite Nanoparticles

Iron oxide magnetic nanoparticles were prepared like so:

50 mg of FeCl₃ 6 H₂O and 50 mg of FeSO₄ were dissolved in H₂O and then treated with NH₄OH (commercial solution 30%), formed particles were collected using a magnet and dissolved in deionised H₂O.

Composite Fe₂O₃-MIP nanoparticles were then prepared as follows:

3.8 mg of N-isopropylacrylamide, 3.3 mg of N-tert-butylacrylamide, 0.58 mg of N-3-aminopropyl methacrylamide hydrochloride, 0.22 μL of acrylic acid and 0.2 mg of NN-methylene bisacrylamide were dissolved in 5 mL of water, this was followed by 300 μL of commercial 50% stock solution of hydrogen peroxide and 50 μL of iron oxide magnetic nanoparticles, prepared as described above.

An assay was then prepared by providing a mixture of bromopyrogallol red (BPR) and the Fe₂O₃-MIP nanoparticles, as follows:

200 uL of Fe₂O₃-MIP nanoparticles, 100 μL of H₂O₂ and 200 μL of bromopyrogallol red solution were used. Samples were added to separate wells and allowed to incubate for 30 minutes. The absorbance was then measured (results not shown).

### Example 8—Investigating the Specificity of Binding Sites on Composite Nanoparticles (Nanozymes) for Use in Homogeneous Assays

Composite nanoparticles with a gold core and MIP shell imprinted with amphetamine (Au/MIP nanozymes) were synthesized using the following protocol: The following monomers were dissolved together in PBS (1 mL, 100 mM, pH 7.5): N-3-aminopropyl meth acrylamide (3 mg, 17 μmol), N-isopropyl acrylamide (19.5 mg, 172 μmol), N-tert-butyl acrylamide solution (400 μL, 236 μM in ethanol), acrylic acid (50 μL, 313 μM in water) and N,N′-methylenebisacrylamide (400 μL, 38 μM in water). The monomer/PBS solution was mixed with 150 μL of gold nanoparticle stock solution (diameter 20 nm, 0.06 μM, in 5 mM PBS, pH 7.5). The monomeric solution was then sonicated for 20 min under nitrogen. 30 g of template glass beads modified with amphetamine were added to the monomeric solution. The polymerization was initiated via addition of hydrogen peroxide (50% w/v, 600 μL, 14.7 mM). The resulting mixture was shaken for 2 h during polymerization. The same steps were used to prepare a control polymer by replacing the amphetamine modified glass beads with blank glass beads (only silanized, with no amphetamine). After polymerization, the solution was discarded and the solid phase was transferred to a Solid Phase Extraction (SPE) cartridge and washed with water at 4° C. (40 mL×2 times). Then, the resulting Au/MIP nanozymes were eluted from the solid phase, first by washing with hot water (60° C., 30 mL×2 times) and then hot ethanol (60° C., 30 mL×2 times). Finally, the Au/MIP nanozymes were dialyzed (72 h), using a 10 kDa cut-off snake skin dialysis membrane and water was changed every 4 hours.

The catalytic activity of the Au/MIP nanozyme is modulated by its binding interaction with the specific target molecule (amphetamine in this case). As explained in Example 6, the analyte binding to the MIP triggers structural changes in the polymer which exposes sections of the gold core available for catalysis.

To illustrate the induced fit phenomena observed for developed nanozymes, their hydrodynamic size was measured in the presence of various targets using DLS (dynamic light scattering). The Au/MIP nanozymes in solution displayed a size of 124.5 ±1.8 nm (PDI, 0.112). Upon exposure to amphetamine, the Au/MIP nanozymes swelled, increasing in size by ~53%. Conversely, in response to the same concentration of paracetamol the size of the Au/MIP nanozymes showed minimal change, with only a ~6% increase in size. This outcome highlights that specific actuation of the nanozyme exclusively occurs in the presence of the target analyte. The control experiments using Au/NIP did not show any significant change (<0.8%).

### Example 9—Investigating the Impact of Gold-Core Diameter in Composite Nanoparticles (Nanozymes) for Use in Homogeneous Assays

Composite nanoparticles with gold cores and MIP shells imprinted with amphetamine were prepared as described in Example 8. Au nanoparticles with varying diameters (5, 20, 50, and 100 nm) were added to the polymeric mixture, thus generating composite nanoparticles (nanozymes) with different diameter gold cores. As a control, a non-imprinted polymer with gold nanoparticles (AuNPs/NIP) was used.

The catalytic properties of synthesised Au/MIP nanozymes and AuNPs/NIP were assessed via a colorimetric assay with BRP. Colorimetric assays with BRP were carried out using the following assay conditions: each well contained 10 μL of the Au/MIP nanozyme (0.4 mg mL⁻¹), 135 μL of 10 nM BPR in PBS (5 mM, pH 7.0), 135 μL of H₂O₂ (50% w/v) and 20 μL of amphetamine (66.7 nM).

Overall, the assay results, which are summarised in Table 1, show that MIP1 and MIP2, with gold cores of diameter 5 nm and 20 nm respectively, exhibited the highest reaction rates for the oxidation of the BPR dye, as demonstrated by the highest Vₘₐₓ values for these particles. These particles also exhibited the greatest response to amphetamine, as demonstrated by these particles having the lowest limit of detection (LOD). Thus, there is clear connection between the size of gold core and the catalytic activity of Au/MIP nanozymes.

TABLE 1
Parameters of synthetised Au/MIP nanozymes
and their assay performance;

| | | Diameter | | | | |
|---|---|---|---|---|---|---|
| | Diameter | of Au/MIP | | | | |
| | of Au | nanozyme | | | | Linear |
| MIP | core | particles | Kₘ | Vₘₐₓ | LOD | range |
| Particle | (nm) | (nm)* | (mM) | (nM/s) | (nM) | (nM) |
| MIP1 | 5 | 98.4 ± | 4.0 | 1.5 × | 0.17 | 0.6-40 |
| | | 2.4 | | 10⁻⁴ | | |
| MIP2 | 20 | 124.5 ± | 3.8 | 1.0 × | 0.23 | 0.6-40 |
| | | 1.8 | | 10⁻⁴ | | |
| MIP3 | 50 | 324.2 ± | 5.5 | 7.0 × | 0.46 | 2.0-40 |
| | | 7.3 | | 10⁻⁵ | | |
| MIP4 | 100 | 726.9 ± | 4.0 | 5.5 × | 0.52 | 10-40 |
| | | 13.6 | | 10⁻⁵ | | |
| NIP | 5 | 89.01 ± | 1.0 | 5.0 × | No | No |
| | | 3.1 | | 10⁻⁵ | response | response |
| | | | | | to the | to the |
| | | | | | target | target |

*asterisk indicates that the nanozyme diameters were determined using dynamic light scattering (DLS).
*The diameter was measured using dynamic light scattering (DLS).

### Example 10—Investigating the Detection Range of a Homogeneous Assay Based on Composite Nanoparticles (Nanozymes)

Composite nanoparticles with 50 nm gold cores and MIP shells imprinted with amphetamine (i.e. MIP3) were prepared as described in Example 8. Colorimetric assays with BRP were carried out using the conditions described in Example 9, but with using varying concentrations of amphetamine (0.3 to 133 nM). The results are shown in FIG. 7.

Importantly, this detection range of the assay falls within a clinically relevant range for forensic applications: an amphetamine concentration ranging from 20 to 100 ng mL⁻¹ (equivalent to 0.148 nM to 0.74 nM) in urine is considered to fall within the therapeutic range (Drugs UNOo, Laboratory C, Section S. Recommended Methods for the Identification and Analysis of Amphetamine, Methamphetamine and Their Ring-substituted Analogues in Seized Materials: Manual for Use by National Drug Testing Laboratories. New York: United Nations (2006)); concentrations surpassing 100 ng mL⁻¹ (0.74 nM) in urine may suggest possible drug abuse; and levels exceeding 2500 ng mL⁻¹ (18.5 nM) can be toxic and potentially life-threatening (Moeller K E, Kissack J C, Atayee R S, Lee K C. Clinical interpretation of urine drug tests: what clinicians need to know about urine drug screens. In: Mayo Clinic Proceedings). Elsevier (2017)).

### Example 11—Investigating the Specificity of Homogeneous Assay Based on Composite Nanoparticles (Nanozymes)

Composite nanoparticles with 5 nm gold cores and MIP shells imprinted with amphetamine (i.e. MIP1) were prepared as described in Example 8. Colorimetric assays with BRP were carried out as described in Example 9 using varying concentrations (0.3 to 40 nM) of amphetamine or other drug. A non-imprinted polymer decorated with gold nanoparticles (AuNPs/NIP) was used as a control.

The assay specificity was first analysed by comparing the responses of Au/MIP and Au/NIP nanozymes to amphetamine. As shown in FIG. 8, no noticeable response to amphetamine was detected for Au/NIP nanozymes. This result demonstrates the specificity of the developed nanozyme assay for a corresponding target.

The assay specificity for amphetamine was further investigated by conducting assays in the presence of different drugs. Separate assays were conducted in the presence of paracetamol, morphine, and cocaine. As shown in FIG. 9, practically no cross-reactivity was observed for these interfering molecules, demonstrating the excellent specificity of Au/MIP nanozymes for a corresponding target.

### Example 12—Homogeneous Assays Based on Composite Nanoparticles for Use in Different Mediums and Storage Conditions

To demonstrate potential applications, the nanozyme assay was employed to detect amphetamine levels in both urine and plasma. Colorimetric assays with BRP were carried out as described in Example 9 using composite nanoparticles with 5 nm gold cores (i.e. MIP1) and varying concentrations (0.3 to 40 nM) of amphetamine. The assays were carried out varying mediums (PBS, plasma and urine).

As shown in FIG. 10, the assay remains operational within these biological matrices. The assay limit of detection (LOD) was determined to be 0.17 nM in buffer, 5.1 nM in urine, and 23.9 nM in plasma.

Additionally, assay performance was measured over 6 months for Au/MIP nanozymes stored in a fridge at 5° C. No noticeable variations in assay performance were observed for nanozymes stored in these conditions (results not shown), indicating excellent stability of synthesised materials.

### Example 13—Detection of o-Nitrophenol in a Gaseous Phase Using LSPR Au Nanochips Coated with MIPs

Localized Surface Plasmon Resonance (LSPR) Au nanochips are glass substrates with randomly distributed gold nanostructures deposited thereon. These Au nanochips were coated with different composite gold MIP nanoparticles (NPs), including 0-nitrophenol, citric acid, picric acid imprinted MIP NPs and blank (non-imprinted) NIP NPs. The composite nanoparticles were synthesized as described in Example 2.

The LSPR Au nanochips then were placed into spectrophotometric cuvettes containing different amounts of the solid explosive analogue analyte 0-nitrophenol and then placed into the working channel of a photometer. An uncoated Au nanochip in a spectrophotometric cuvette was placed in the reference channel of the photometer. The cuvettes in both channels were closed with caps during the measurements in order to reduce the background noise.

Kinetic dependences of absorbance shift for the Au nanochips upon exposure to 0-nitrophenol vapor from different amounts of the explosive analogue (powder samples) were recorded. The absorbance responses expressed concentration-dependent behavior and a signal-to-noise ratio sufficient for the detection of femtogram amounts of explosive analogue analyte (10 femtogram of 0-nitrophenol in 4.5 cm³ volume). The optical absorbance response time was up to 5 min.

The inventors observed that the Au nanochip coated with 0-nitrophenol imprinted MIP NPs exhibited a cumulative dose-response.

### Example 14—Detection of o-Nitrophenol in Gaseous Phase Using LSPR Au Nanochips Coated with Composite Nanoparticles in a ‘Real-Life’ Scenario

A real-life scenario for the detection of trace amount of explosive analogue on a surface of interest was considered.

To prepare a ‘real-life’ model of a trace amount of explosive analogue, the following protocol was used: 1 femtogram 0-nitrophenol was dissolved in 10 μl 2-propanol, drop casted on a glass surface and allowed to dry completely for 1 minute. A clean dry cotton swab was then used to rub the dried drop and it was put into a spectrophotometric cuvette. An Au nanochip was provided which was coated with composite nanoparticles where the MIPs were imprinted with 0-nitrophenol (prepared as described in Example 13). The Au nanochip was placed into spectrophotometric cuvette containing the cotton swab. The cuvette was closed with a cap and measurement taken using a two-channel photometer.

The kinetic dependence of absorbance shift for the coated Au nanochip upon exposure to 0-nitrophenol vapor from the dry cotton swab is shown in FIG. 11. The absorbance response clearly shows an increase when a trace amount of 0-nitrophenol is present near the Au nanochip, thus confirming capability to detect of ~1 femtogram of an explosive analogue in a real-life scenario with a response time of minutes and an optical absorbance response up to 5·10⁻⁴ in magnitude.

### Example 15—Design of an Optical Fibre-Based Detection System

The inventors developed an experimental set-up designed to reduce detector cost, while retaining very low limits-of-detection (LOD) and selectivity towards the target substances which was found using the LSPR photometer detection system described in Examples 13 and 14.

The detector is based on optical fibres coupled to a portable spectrometer, as shown in FIG. 12. The detector comprises a light source 3, e.g. a USB powered LED, a sensing element 4 comprising an optical fibre with composite nanoparticles deposited thereon, an optical detector 2, e.g. a portable spectrometer or a photodiode, and an operating device 1, e.g. a laptop. Light is emitted from the light source, propagates in the optical fibre, and is collected by the optical detector. The composite gold MIP nanoparticles serve as a sensing element configured to detect a target substance 5. The spectra of the Au core changes upon binding of a target molecule to the MIP.

This detection system was manufactured by coating the tapered portion of an optical fibre with gold nanoparticles. MIP coatings were then synthesised onto the gold nanoparticles.

The detection system was tested for its capability to detect a target substance, cocaine. FIG. 13 shows the optical signal generated in this test and it can be seen that there is an increase in intensity upon exposure of the sensor to the target substance.

As demonstrated by FIG. 13, the target molecule will interact with the composite nanoparticles. The interaction between the target molecule and the composite nanoparticles has a direct and proportional effect on the propagated optical signal. The optical detector then records this signal change to indicate the presence and/or concentration of the target molecule.

Use of optical fibres in place of Au nanochips allows for a significant reduction in costs while retaining low limits of detection. In comparison to LSPR as used in the photometer detector in Examples 13 and 14, this technique has the advantage of being more resistant to interference.

### Methods

### Preparation of Glass Beads

For the solid phase synthesis, glass beads were functionalized with amphetamine. For that, 60 g of glass beads were incubated in amphetamine (65 mg, 0.481 μmol) in borate buffer (65 mL, 100 mM, pH 9.2) for 8 h, protected from light. In order to block unreacted iodine groups, mercaptoethanol (10 μL, 0.143 μmol) of was added to the mixture and incubated for 2 h. Subsequently, the glass beads were washed (100 mL×2 times) with water and then acetone (100 mL×4 times) and then dried under vacuum.

### Development of BPR Assay

Corning® 96-wells microplates (300 μL well volume) and a Hidex Sense 425-301 microplate reader were employed for measurements. Nanozyme catalytic activity was evaluated using the colorimetric assay format, measuring the absorbance change due to the oxidation of Bromo pyrogallol red (BPR, a chromogenic substrate) by hydrogen peroxide (H₂O₂), similar to other peroxidase-like activity assays.

1. A method of producing a composite particle, the method comprising contacting a catalyst particle, an oxidant and a plurality of monomers, wherein the oxidant is a peroxide, a persulfate or ozone, and the catalyst particle is or comprises a material which possesses Fenton or Fenton-like catalytic activity, such that the catalyst particle catalyses the disproportionation of the oxidant to create an oxygen-radical species, which then initiates a polymerisation reaction and thereby cause the plurality of monomers to polymerise, and thereby form a polymeric shell around the catalyst particle.

2. The method of claim 1, wherein the method comprises contacting the catalyst particle, the oxidant and the plurality of monomers in a solution and/or wherein the composite particle is a nanoparticle and the catalyst particle is a nanoparticle.

3. (canceled)

4. The method of claim 1, wherein the catalyst particle is unbound or wherein the catalyst particle is bound to a substrate, and preferably wherein the substrate is an optical fibre comprising a polymer coating.

5. (canceled)

6. The method of claim 1, wherein the catalyst particle comprises or consists of a metal, a metal alloy, a metal oxide, a metal salt, a semiconductor, a two-dimensional material, a fullerene, a carbon nanotube, a quantum dot, a carbon nanodot, a carbide and/or a nitride, optionally wherein the catalyst particle comprises or consists of a metal or a metal oxide, and the metal is gold, silver, platinum, palladium, iron, nickel, zinc, chromium, titanium, zirconium, hafnium, vanadium, niobium, tantalum, molybdenum or tungsten, and the metal oxide is iron oxide, aluminium oxide, titanium oxide, cerium oxide, zirconium oxide, hafnium oxide, vanadium oxide, niobium oxide, tantalum oxide, chromium oxide, molybdenum oxide or tungsten oxide.

7. (canceled)

8. The method of claim 1, wherein the method comprises producing the catalyst particle and contacting the catalyst particle, the oxidant and the plurality of monomers in a single step, such that the method comprises contacting a catalyst particle precursor, the oxidant and the plurality of monomers, wherein the catalyst particle precursor is configured to produce the catalyst particle, optionally wherein the catalyst particle precursor comprises a reagent which can be oxidised, reduced or hydrolysed to produce the catalyst particle; and an oxidising agent, a reducing agent or a hydrolysing agent, optionally wherein the reagent which can be oxidised, reduced or hydrolysed to produce the catalyst particle is an inorganic salt, an acid or a hydroxide, and the inorganic salt, the acid or the hydroxide comprises a metal cation.

9. (canceled)

10. (canceled)

11. The method of claim 1, wherein the method comprises contacting the catalyst particle, oxidant and plurality of monomers in the presence of a template species, such that the polymeric shell is or comprises a molecular imprinted polymer (MIP).

12. A composite particle produced by the method of claim 1.

13. (canceled)

14. A method of sensing a target molecule in a solution, the method comprising:
contacting the composite particle of claim 35 with a solution; and
sensing an optical property of the catalyst particle, and thereby sensing the target molecule in the solution, optionally wherein the optical Property is surface plasmon resonance (SPR) absorbance, localised surface plasmon resonance (LSPR) absorbance or fluorescence.

15. (canceled)

16. A method of sensing a target molecule in a solution, the method comprising:
disposing an electrode with a the composite particle of claim 35 immobilised thereon, and a further electrode in a solution;
applying a voltage across the electrodes, optionally wherein applying the voltage across the two electrodes and measuring the current comprises applying differential pulse voltammetry (DPV); and
measuring the current, to thereby sensing the target molecule in the solution.

17. (canceled)

18. (canceled)

19. A lateral flow sensor comprising the composite particle of claim 35.

20. A method of sensing a target molecule in a sample, the method comprising contacting the sample with the lateral flow sensor of claim 19 and observing whether or not a test line appears on the lateral flow sensor, and thereby sensing the target molecule in a sample.

21. A method of sensing a target molecule in a sample, the method comprising:
contacting a spectrophotometric reagent, the composite particle of claim 35, an oxidising or reducing agent and the sample; and
measuring the absorbance of the resultant composition, to thereby sensing the target molecule in the sample;
optionally wherein the spectrophotometric reagent is a redox sensitive dye.

22. (canceled)

23. (canceled)

24. A pharmaceutical composition comprising the composite particle of claim 35 and a pharmaceutically acceptable carrier.

25. (canceled)

26. (canceled)

27. (canceled)

28. The composite particle of claim 35, wherein the composite particle comprises a drug molecule adsorbed therein and/or wherein the composite particle comprises an antibody or an aptamer conjugated to the polymeric shell, or the polymeric shell comprises an MIP, and the antibody, aptamer or MIP is configured to deliver the composite particle to a specific target.

29. The composite particle of claim 35, wherein the catalyst particle comprises a magnetic material.

30. (canceled)

31. A sensor comprising:
a substrate;
a plurality of nanoparticles disposed on the substrate; and
a plurality of composite particles of claim 35 disposed on the substrate.

32. A method of sensing a target molecule in a fluid, the method comprising:
providing the sensor of claim 31;
contacting the sensor with a fluid; and
sensing an optical property of the sensor, and thereby sensing the target molecule in the fluid.

33. A sensor comprising
an optical fibre extending between a first end and a second end, the optical fibre comprising a sensing portion, wherein the sensing portion is configured to allow generation of an evanescent field; and
the composite particle of claim 35 disposed on the sensing portion of the optical fibre, the composite particle comprising a polymeric shell around a catalyst particle.

34. A method of sensing a target molecule in a fluid, the method comprising:
providing the sensor of claim 33;
contacting the composite particle with the fluid;
inputting an optical signal into the first end of the optical fibre, and thereby generating an evanescent field adjacent the sensing portion of the optical fibre, such that the composite particle is at least partially disposed in the evanescent field; and
detecting an output optical signal or value from the second end of the optical fibre and comparing the output optical signal to a reference optical signal or value to thereby sense the target molecule in the fluid.

35. A composite particle, wherein the composite particle comprises a polymeric shell around a catalyst particle.
